(19) United States
(12) Patent Application Publication
(10) Pub. No.: US 2025/0269011 A1
(43) Pub. Date: Aug. 28, 2025

(54) TREATMENT OF CANCER USING MRNA-MEDIATED VIRUS VACCINATION ANTIGENS DELIVERED BY ATTENUATED BACTERIA

(71) Applicants: Christopher Bradley, Brooklyn, NY (US); Claudia Gravekamp, New York, NY (US)

(72) Inventors: Christopher Bradley, Brooklyn, NY (US); Claudia Gravekamp, New York, NY (US)

(21) Appl. No.: 19/118,076

(22) PCT Filed: Jan. 26, 2022
(86) PCT No.: PCT/US22/13949

§ 371 Date: Apr. 3, 2025

(51) Int. Cl. A61K 39/215; A61K 31/7068; A61K 39/00; A61P 35/00; C12N 7/00; C12N 15/74

(52) U.S. Cl. CPC A61K 39/215; A61K 31/7068; A61P 35/00; C12N 7/00; C12N 15/74; A61K 2039/522; A61K 2039/523; A61K 2039/54; A61K 2039/55561; C12N 2770/20022; C12N 2770/20034

(57) ABSTRACT

Disclosed are methods of treating tumors in a subject including but not limited to reducing the size of a primary tumor, preventing progression of a tumor to metastasis. and reducing the extent of metastasis: comprising administering to the subject an attenuated bacteria that expresses an mRNA-mediated virus vaccination antigen in an amount effective to treat the tumor. Further disclosed are methods of preventing infection of cells in a subject by a virus and/or preventing or reducing in the subject symptoms of a disease caused by the virus: comprising administering to the subject an attenuated bacteria that expresses an mRNA-mediated virus vaccination antigen in an amount effective to protect the subject against the virus and/or its effects. Further disclosed are pharmaceutical compositions, cancer immunotherapy compounds. virus vaccines and therapeutic cancer vaccines comprising an attenuated bacteria that expresses an mRNA-mediated virus vaccination antigen, and methods of developing the same.

### CROSS-REFERENCE TO RELATED APPLICATIONS

This application claims the benefit of U.S. Provisional Patent Application No. 63/141,933, filed Jan. 26, 2021, the contents of which are hereby incorporated by reference herein.

### FIELD OF THE INVENTION

The present invention relates generally to treatments for cancer and protection against viruses, and more particularly to pharmaceutical compositions, cancer immunotherapy compounds, virus vaccines and therapeutic cancer vaccines that comprise, and methods of treating tumors in a subject and protecting a subject against viruses comprising administering to the subject, an attenuated bacteria that expresses an mRNA-mediated virus vaccination antigen in an amount effective to treat the tumor and protect the subject against the virus and/or its effects, and methods of developing the same.

### BACKGROUND OF THE INVENTION

Cancer remains a major health concern worldwide. Success of cancer immunotherapy is hindered by three major problems. a first problem is that tumor-associated antigens (TAAs), used in therapeutic cancer vaccines, are often self-antigens that are overexpressed or mutated in tumor cells compared to normal cells. The T cells in the thymus have been taught earlier in life not to react to self-antigens, and therefore it is difficult to induce strong T cell responses to TAAs. A second problem is that most cancer patients are old, and the elderly react less efficiently than young adults to traditional therapeutic cancer vaccines. This is often due to a lack of naive T cells (which are generated only at a young age, and are used during life) that react for the first time to a new antigen and are responsible for the generation of memory T cells upon repeated exposures with the same antigen. A third problem is that cancer is constantly subject to mutational pressure, which causes the TAAs to not be as useful as the cancer constantly changes.

In addition, old and new viruses remain a threat. Traditional virus vaccines have been shown to be effective, but less so for older subjects. Virus vaccines train the immune system to recognize the disease-causing part of a virus. Traditional virus vaccines contain either weakened viruses or purified signature proteins of viruses, and have enjoyed success in the effective elimination of certain viruses, especially when administered at a young age. While traditional virus vaccines can also be effective when administered at an old age, the effectiveness is reduced in an older host (e.g., due to a lack of naive T cells, as described above).

An added danger is that traditional cancer treatments, such as chemotherapy or other uses of chemotherapeutic agents, can weaken a person's immune system. Therefore, subjects with cancer who are undergoing traditional cancer treatments are more susceptible to viruses, and traditional virus vaccines are less effective in older subjects, which comprises a large percentage of cancer patients.

The present invention addresses these problems and the need for improved treatments for cancers and improved protection against viruses.

### SUMMARY OF THE INVENTION

The present invention provides methods of treating tumors in a subject including but not limited to reducing the size of a primary tumor, preventing progression of a tumor to metastasis, and reducing the extent of metastasis; comprising administering to the subject an attenuated bacteria that expresses an mRNA-mediated virus vaccination antigen in an amount effective to treat the tumor.

The present invention further provides methods of preventing infection of cells in a subject by a virus and/or preventing or reducing in the subject symptoms of a disease caused by the virus; comprising administering to the subject an attenuated bacteria that expresses an mRNA-mediated virus vaccination antigen in an amount effective to protect the subject against the virus and/or its effects.

The present invention further provides methods of (1) treating tumors in a subject including but not limited to reducing the size of a primary tumor, preventing progression of a tumor to metastasis, and reducing the extent of metastasis; and (2) preventing infection of cells in a subject by a virus and/or preventing or reducing in the subject symptoms of a disease caused by the virus; comprising administering to the subject an attenuated bacteria that expresses an mRNA-mediated virus vaccination antigen in an amount effective to treat the tumor and protect the subject against the virus and/or its effects.

The present invention further provides pharmaceutical compositions, cancer immunotherapy compounds, virus vaccines and therapeutic cancer vaccines comprising an attenuated bacteria that expresses an mRNA-mediated virus vaccination antigen, and methods of developing the same.

### Methods

In preferred embodiments, the present invention provides a method of treating a subject, comprising administering to the subject an attenuated bacteria that expresses an mRNA-mediated virus vaccination antigen in an amount effective to treat the subject.

Preferably, in certain embodiments, the mRNA-mediated virus vaccination antigen is an epitope of a protein of a virus, the protein being a protein targeted by an mRNA-mediated vaccine against the virus.

Preferably, in certain embodiments, the mRNA-mediated virus vaccination antigen is an epitope of the spike protein of the SARS-CoV-2 virus.

Preferably, in certain embodiments, treating the subject includes treating a tumor in the subject and/or reducing or preventing metastasis of a tumor in the subject, and the amount effective to treat the subject is an amount effective to treat the tumor and/or reduce or prevent metastasis of the tumor.

Preferably, in certain embodiments, treating the subject includes protecting the subject against a virus and the amount effective to treat the subject is an amount effective to prevent infection by the virus and/or eliminate or reduce symptoms of an infection by the virus. Further preferably, in certain embodiments, treating the subject further includes treating a tumor in the subject and/or reducing or preventing metastasis of a tumor in the subject and the amount effective to treat the subject is also an amount effective to treat the tumor and/or reduce or prevent metastasis of the tumor. Further preferably, in certain embodiments, the tumor is a tumor of one or more of the pancreas, ovary, uterus, neck, head, breast, prostate, liver, lung, kidney, neurones, glia, colon, testicle, or bladder. Further preferably, in certain embodiments the tumor is an inoperable tumor.

Preferably, in certain embodiments, the bacteria is one or more of Listeria monocytogenes, Salmonella thyphimurium, Vibrio cholera, Clostridium, and Bifidobacterium breve.

Preferably, in certain embodiments, prior to administration to the subject, the bacteria are cultured in yeast medium.

Preferably, in certain embodiments, the method further comprises administering CpG to the subject.

Preferably, in certain embodiments, prior to administration of bacteria to the subject, the subject is screened for their major histocompatibility complex (MHC) 1 haplotype and administered an antigen for which the subject shows a CD8 T cell recall response.

Preferably, in certain embodiments, prior to administration of bacteria to the subject, an epitope of the antigen is administered to the subject to generate memory T cells to the antigen.

Preferably, in certain embodiments, bacteria are administered systemically to the subject.

Preferably, in certain embodiments, bacteria are administered by direct injection to a site in the subject.

Preferably, in certain embodiments, bacteria are administered in myeloid-derived suppressor cells (MDSCs).

Preferably, in certain embodiments, the method further comprises administering to the subject a chemotherapeutic agent that reduces the number of myeloid-derived suppressor cells (MDSCs). Further preferably, the chemotherapeutic agent is gemcitabine.

Preferably, in certain embodiments, the subject is a mammal.

Preferably, in certain embodiments, the subject is a human.

### Compositions, Compounds, Virus Vaccines and Cancer Vaccines

In preferred embodiments, the present invention provides, for use in treating a subject, one or more of the following comprising an attenuated bacteria that expresses an mRNA-mediated virus vaccination antigen in an amount effective to treat the subject: pharmaceutical composition, immunotherapy compound, virus vaccine and therapeutic cancer vaccine.

Preferably, in certain embodiments, the mRNA-mediated virus vaccination antigen is an epitope of a protein of a virus, the protein being a protein targeted by an mRNA-mediated vaccine against the virus.

Preferably, in certain embodiments, the mRNA-mediated virus vaccination antigen is an epitope of the spike protein of the SARS-CoV-2 virus.

Preferably, in certain embodiments, treating the subject includes treating a tumor in the subject and/or reducing or preventing metastasis of a tumor in the subject, and the amount effective to treat the subject is an amount effective to treat the tumor and/or reduce or prevent metastasis of the tumor.

Preferably, in certain embodiments, treating the subject includes protecting the subject against a virus and the amount effective to treat the subject is an amount effective to prevent infection by the virus and/or eliminate or reduce symptoms of an infection by the virus. Further preferably, in certain embodiments, treating the subject further includes treating a tumor in the subject and/or reducing or preventing metastasis of a tumor in the subject and the amount effective to treat the subject is also an amount effective to treat the tumor and/or reduce or prevent metastasis of the tumor. Further preferably, in certain embodiments, the tumor is a tumor of one or more of the pancreas, ovary, uterus, neck, head, breast, prostate, liver, lung, kidney, neurones, glia, colon, testicle, or bladder. Further preferably, in certain embodiments the tumor is an inoperable tumor.

Preferably, in certain embodiments, the bacteria is one or more of Listeria monocytogenes, Salmonella thyphimurium, Vibrio cholera, Clostridium, and Bifidobacterium breve.

Preferably, in certain embodiments, prior to administration to the subject, the bacteria are cultured in yeast medium.

Preferably, in certain embodiments, treating the subject includes administering CpG to the subject.

Preferably, in certain embodiments, prior to administration of bacteria to the subject, the subject is screened for their major histocompatibility complex (MHC) 1 haplotype and administered an antigen for which the subject shows a CD8 T cell recall response.

Preferably, in certain embodiments, treating the subject includes, prior to administration of bacteria to the subject, administering to the subject an epitope of the antigen to generate memory T cells to the antigen.

Preferably, in certain embodiments, treating the subject includes administering the bacteria systemically to the subject.

Preferably, in certain embodiments, treating the subject includes administering the bacteria by direct injection to a site in the subject.

Preferably, in certain embodiments, treating the subject includes administering the bacteria in myeloid-derived suppressor cells (MDSCs).

Preferably, in certain embodiments, treating the subject includes administering to the subject a chemotherapeutic agent that reduces the number of myeloid-derived suppressor cells (MDSCs). Further preferably, the chemotherapeutic agent is gemcitabine.

Preferably, in certain embodiments, the subject is a mammal.

Preferably, in certain embodiments, the subject is a human.

### Arrangement in in a Tumor Microenvironment

In preferred embodiments, the present invention provides an arrangement, in a tumor microenvironment in a subject, of a cancer immunotherapy compound and a chemotherapeutic compound, the cancer immunotherapy compound having exited a first myeloid-derived suppressor cell in the tumor microenvironment and entering a tumor cell in the tumor microenvironment, the chemotherapeutic compound causing cell death in a second myeloid-derived suppressor cell in the tumor microenvironment, the cancer immunotherapy compound comprising: an attenuated bacteria configured to induce an immune response in the subject to the tumor cell substantially at least equal in magnitude to an immune response in the subject to a pathogen recognized by the immune system of the subject, the attenuated bacteria configuration including having as a payload a fusion of a truncated non-cytolytic Listeriolysin-O and a non-self external antigen of the pathogen, the payload lacking a cancer specific antigen, the Listeriolysin-O including a signal sequence for inducing the tumor cell to present the non-self external antigen on an external surface of the tumor cell, wherein an intraperitoneal injection of the cancer immunotherapy compound into the subject permits survival of the cancer immunotherapy compound until the attenuated bacteria infects the first myeloid-derived suppressor cell, travel of the first myeloid-derived suppressor cell to the tumor microenvironment, and subsequent exit, facilitated by the Listeriolysin-O, of the cancer immunotherapy compound from the first myeloid-derived suppressor cell, delivers the cancer immunotherapy compound to the tumor microenvironment, infection of the tumor cell by the cancer immunotherapy compound, facilitated by immune suppression in the tumor microenvironment caused at least in part by the second myeloid-derived suppressor cell prior to the cell death, and subsequent secretion by the cancer immunotherapy compound of the payload in the tumor cell causes the tumor cell, in response to the signal sequence, to present the non-self external antigen on the external surface of the tumor cell, recognition of the non-self external antigen by the immune system causes the immune response to the tumor cell, and the immune response is enhanced by a reduction of immune suppression in the tumor microenvironment caused by the cell death caused by the chemotherapeutic compound.

Preferably, in certain embodiments, the attenuated bacteria is an attenuated strain of one or more of Listeria monocytogenes, Salmonella thyphimurium, Vibrio cholera, Clostridium, and Bifidobacterium breve.

Preferably, in certain embodiments, the non-self external antigen is an epitope of a protein of a virus, the protein being a protein targeted by an mRNA-mediated vaccine against the virus.

Preferably, in certain embodiments, the mRNA-mediated virus vaccination antigen is an epitope of the spike protein of the SARS-CoV-2 virus.

Preferably, in certain embodiments, the chemotherapeutic compound is gemcitabine.

Preferably, in certain embodiments, the subject is a target subject and the arrangement includes, with regard to an amount of the cancer immunotherapy compound and an amount of the chemotherapeutic compound being clinically effective for a mouse subject, the corresponding clinically effective respective amounts of the cancer immunotherapy compound and the chemotherapeutic compound for the target subject.

Throughout this application various publications are referred to in parentheses or superscript. Full citations for these references may be found at the end of the specification. The disclosures of these publications are hereby incorporated by reference in their entirety into the subject application to more fully describe the art to which the subject invention pertains.

### BRIEF DESCRIPTION OF THE DRAWINGS

Documents at the citations included herein are hereby incorporated by reference in their entirety into the subject application.

FIG. 1 illustrates the SARS-CoV-2 genome. Image By Jonathan Corum, New York Times. Data Source: Preliminary genomic characterisation of an emergent SARS-CoV-2 lineage in the UK defined by a novel set of spike mutations Report written by: Andrew Rambaut1, Nick Loman2, Oliver Pybus3, Wendy Barclay4, Jeff Barrett5, Alesandro Carabelli6, Tom Connor7, Tom Peacock4, David L Robertson8, Erik Volz4, on behalf of COVID-19 Genomics Consortium UK (CoG-UK)9.

FIG. 2 illustrates a pET-DEST42 plasmid vector.

FIG. 3 illustrates the spike protein sequence of the SARS-CoV-2 virus ready for insertion into the pDEST42 plasmid. Relevant citations: Wrapp, Daniel, et al. “Cryo-EM structure of the 2019-nCoV spike in the prefusion conformation.” Science 367.6483 (2020): 1260-1263.

FIG. 4 describes identified mouse T cell epitopes of the spike protein. Citations mentioned: 1) Grifoni, Alba et al., A Sequence Homology and Bioinformatic Approach Can Predict Candidate Targets for Immune Responses to SARS-CoV-2, Cell Host & Microbe, Volume 27, Issue 4, 671-680.e2 2) Zhang B Z, Hu Y F, Chen L L, Yau T, Tong Y G, Hu J C, Cai J P, Chan K H, Dou Y, Deng J, Wang X L, Hung I F, To K K, Yuen K Y, Huang J D. Mining of epitopes on spike protein of SARS-CoV-2 from COVID-19 patients. Cell Res. 2020 August; 30(8):702-704. doi: 10.1038/s41422-020-0366-x. Epub 2020 Jul. 1. PMID: 32612199; PMCID: PMC7327194.

FIG. 5 describes identified human T cell epitopes of the spike protein. Citations mentioned: 1) Grifoni, Alba et al., A Sequence Homology and Bioinformatic Approach Can Predict Candidate Targets for Immune Responses to SARS-CoV-2, Cell Host & Microbe, Volume 27, Issue 4, 671-680.e2 2) Ethan Fast, Russ B. Altman, Binbin Chen, Potential T-cell and B-cell Epitopes of 2019-nCoV, bioRxiv 2020.02.19.955484; doi: https://doi.org/10.1101/2020.02.19.955484.

FIG. 6 illustrates and describes a Listeria-Spike construct of a preferred embodiment of the present invention.

FIG. 7 illustrates and describes a cascade of events of a preferred embodiment of the present invention.

FIG. 8 illustrates and describes restriction sites in the spike protein.

FIG. 9 illustrates and describes a pCR2.1 vector (Source: Invitrogen).

FIG. 10 illustrates and describes a pCR2.1-XHoI-Spike-Myc-XmaI DNA fragment.

FIG. 11 illustrates and describes a pGG34 plasmid.

FIG. 12 illustrates a Listeria background strain XFL-7.

FIG. 13 illustrates an example protocol for treating subjects with Listeria-Spike, according to a preferred embodiment of the present invention.

FIGS. 14A-D illustrate and describe effects of low versus high doses of Listeria.

### DETAILED DESCRIPTION OF THE INVENTION

### Overview

### Treating Tumors

The present invention provides methods of treating tumors in a subject, including but not limited to reducing the size of a primary tumor, preventing progression of a tumor to metastasis, and reducing the extent of metastasis; comprising administering to the subject an attenuated bacteria that expresses an mRNA-mediated virus vaccination antigen in an amount effective to treat the tumor.

### Virus Protection

The present invention further provides methods of preventing infection of cells in a subject by a virus and/or preventing or reducing symptoms of a disease in the subject caused by the virus; comprising administering to the subject an attenuated bacteria that expresses an mRNA-mediated virus vaccination antigen in an amount effective to protect the subject against the virus and/or its effects.

### Combined Tumor Treatment and Virus Protection

The present invention further provides methods of (1) treating tumors in a subject including but not limited to reducing the size of a primary tumor, preventing progression of a tumor to metastasis, and reducing the extent of metastasis; and (2) preventing infection of cells in a subject by a virus and/or preventing or reducing symptoms of a disease in the subject caused by the virus; comprising administering to the subject an attenuated bacteria that expresses an mRNA-mediated virus vaccination antigen in an amount effective to treat the tumor and protect the subject against the virus and/or its effects.

### Compositions, Compounds and Vaccines

The present invention further provides pharmaceutical compositions, cancer immunotherapy compounds and therapeutic cancer vaccines comprising an attenuated bacteria that expresses an mRNA-mediated virus vaccination antigen, and methods of developing the same.

### Terms and Examples

The subject can be a mammal. In different embodiments, the mammal is a mouse, rat, cat, dog, horse, donkey, mule, sheep, goat, cow, steer, bull, livestock, primate, monkey, or preferably a human. The human can be of different ages, or any age.

The bacteria can be, for example, one of more of Listeria monocytogenes, Salmonella thyphimurium, Vibrio cholera, Clostridium, and Bifidobacterium breve. In a preferred embodiment, the bacteria are Listeria monocytogenes, referred to also herein simply as Listeria. The bacteria are preferably attenuated to reduce or eliminate virulence. As used herein, attenuated Listeria, for example, is denoted as Listeria^{at}.

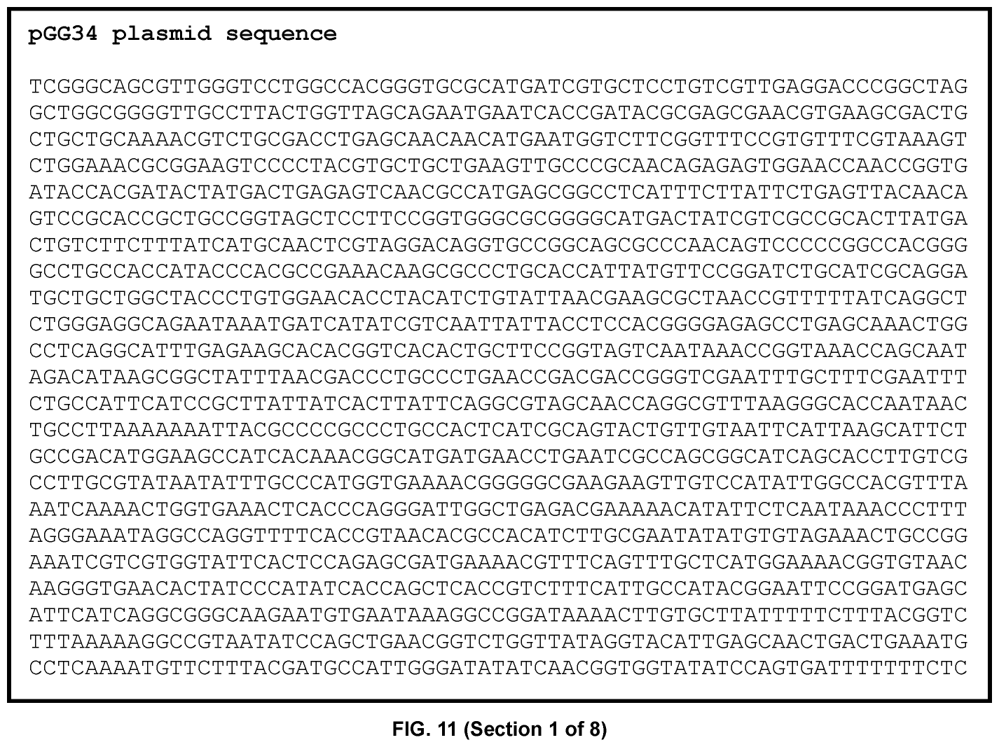

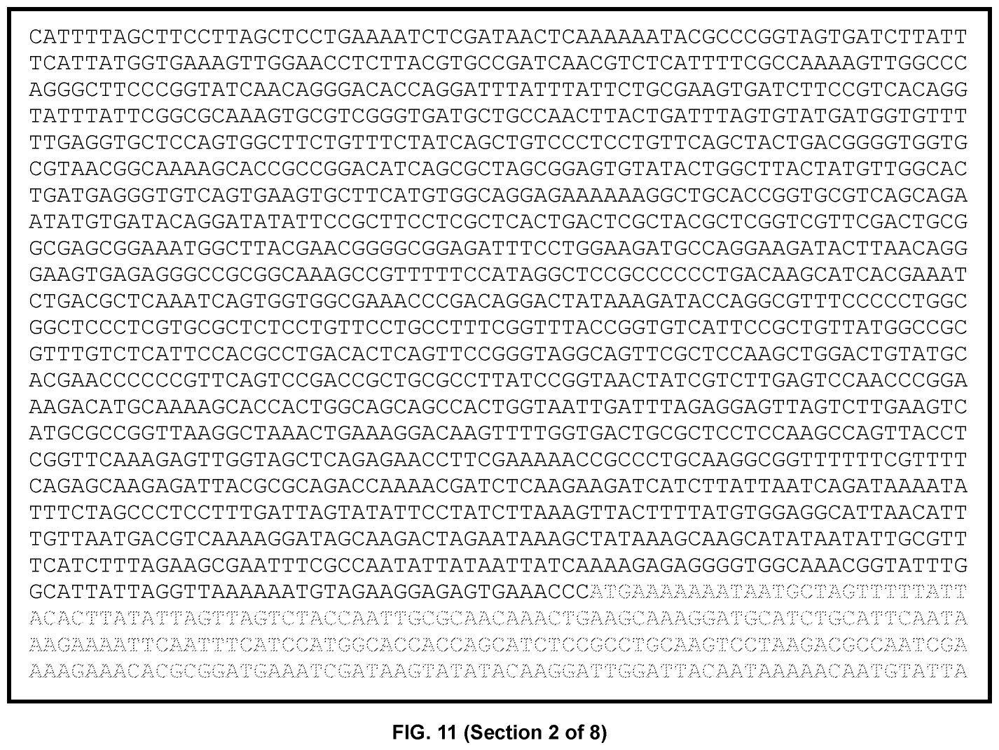

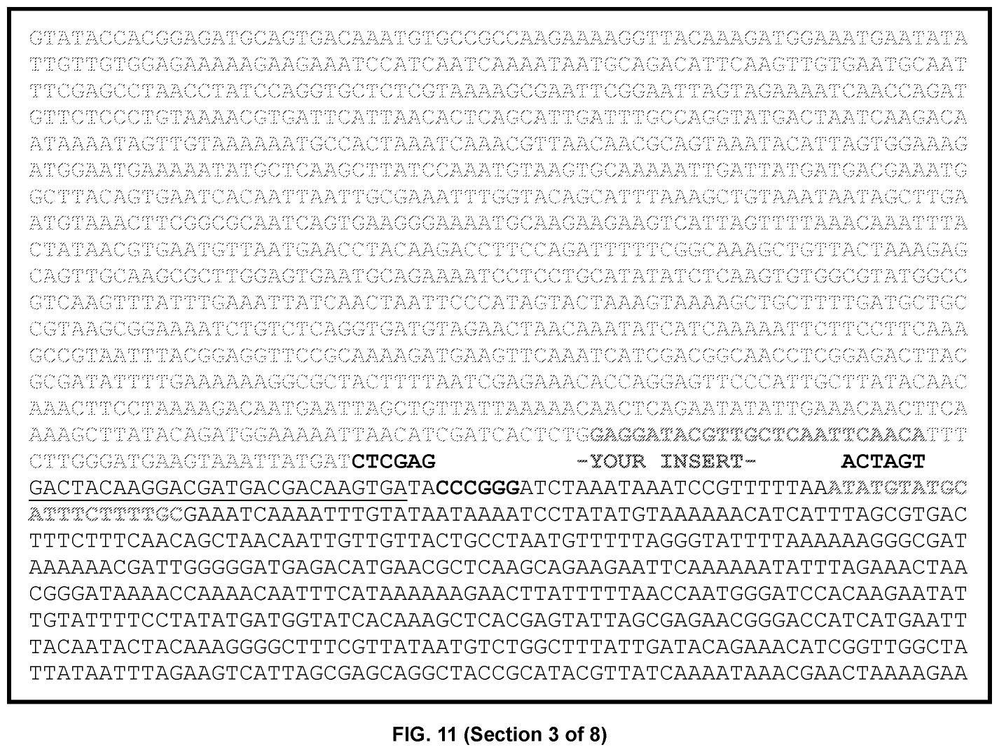

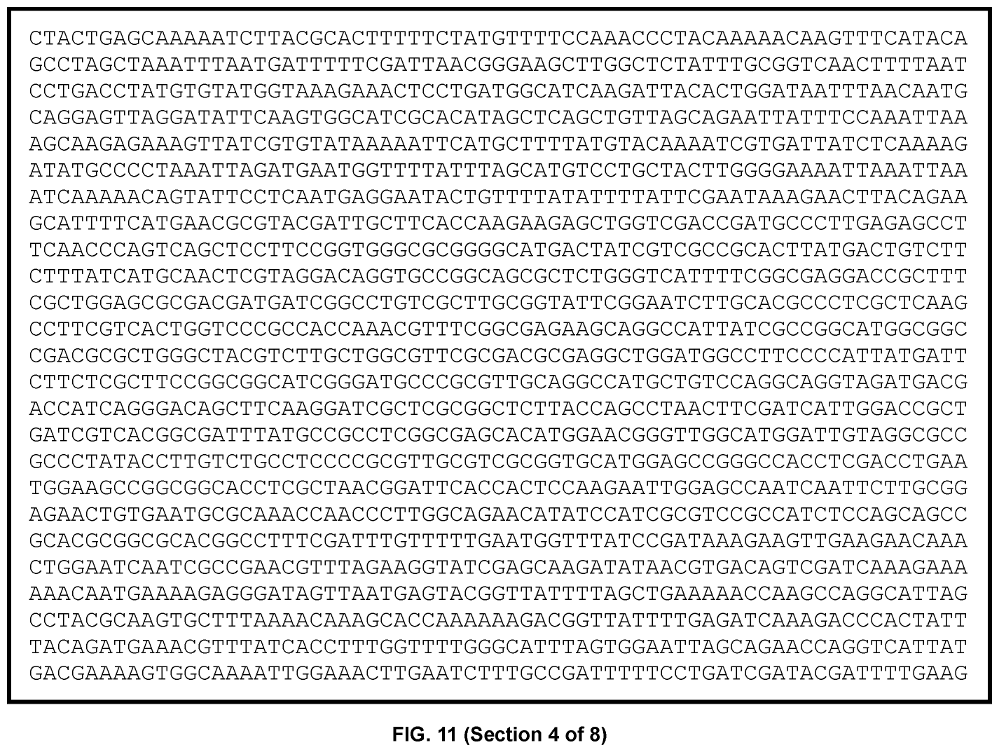

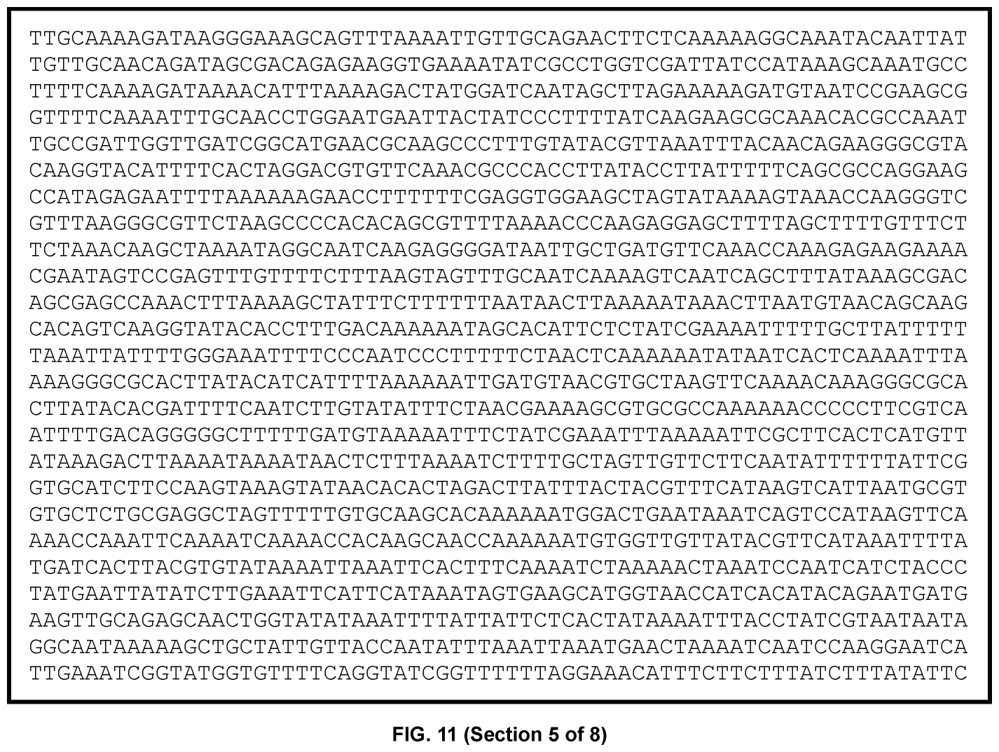

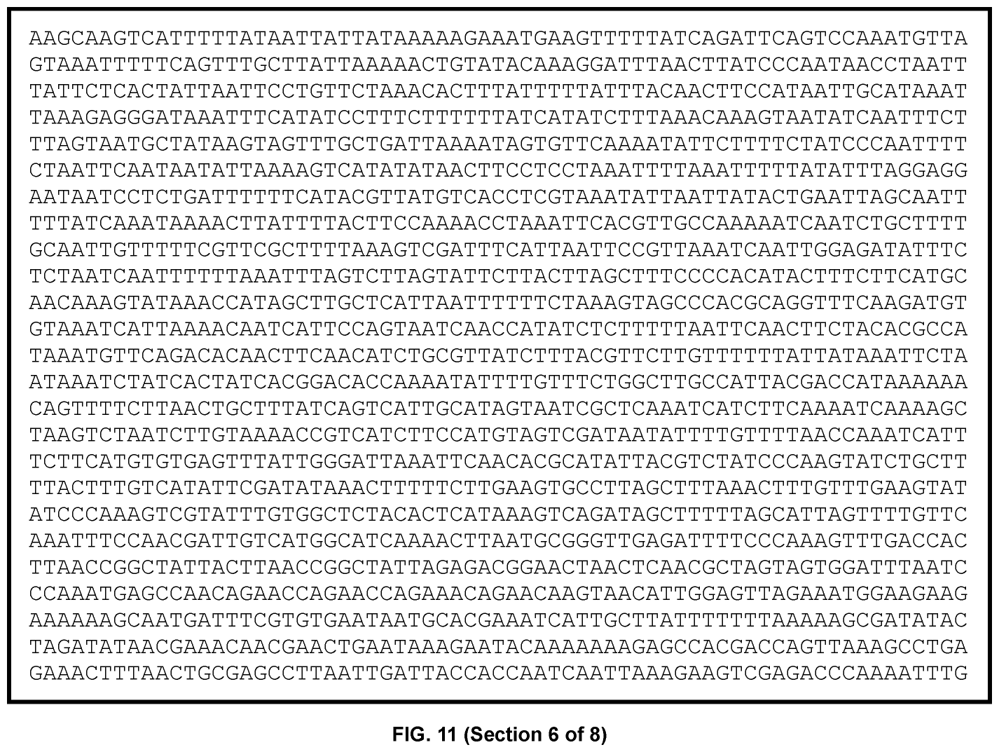

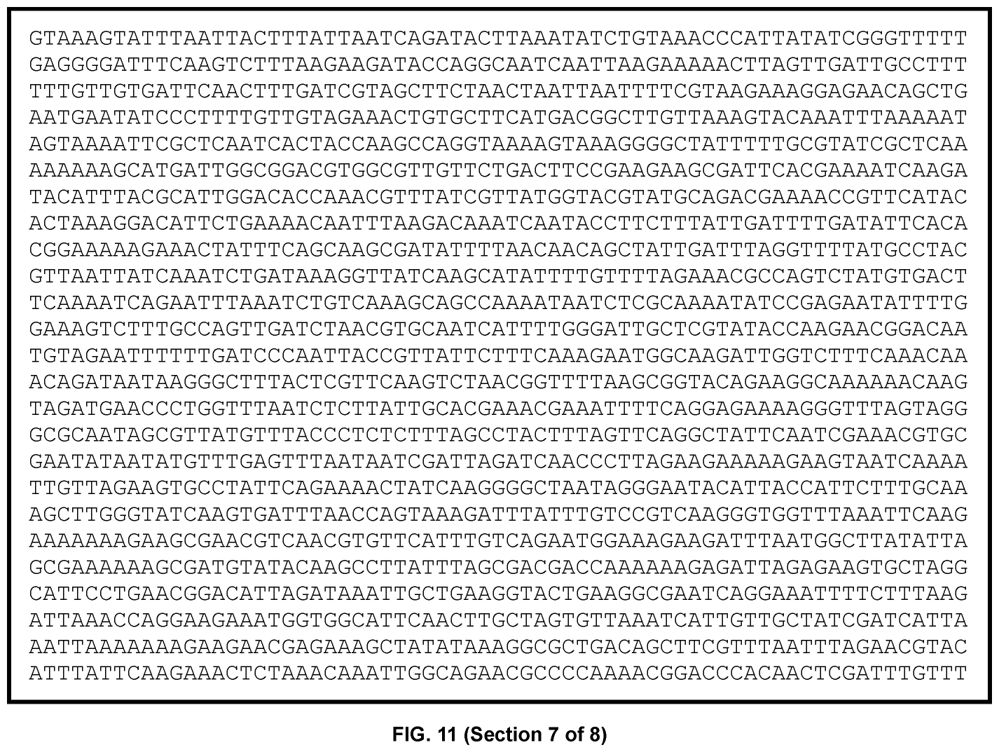

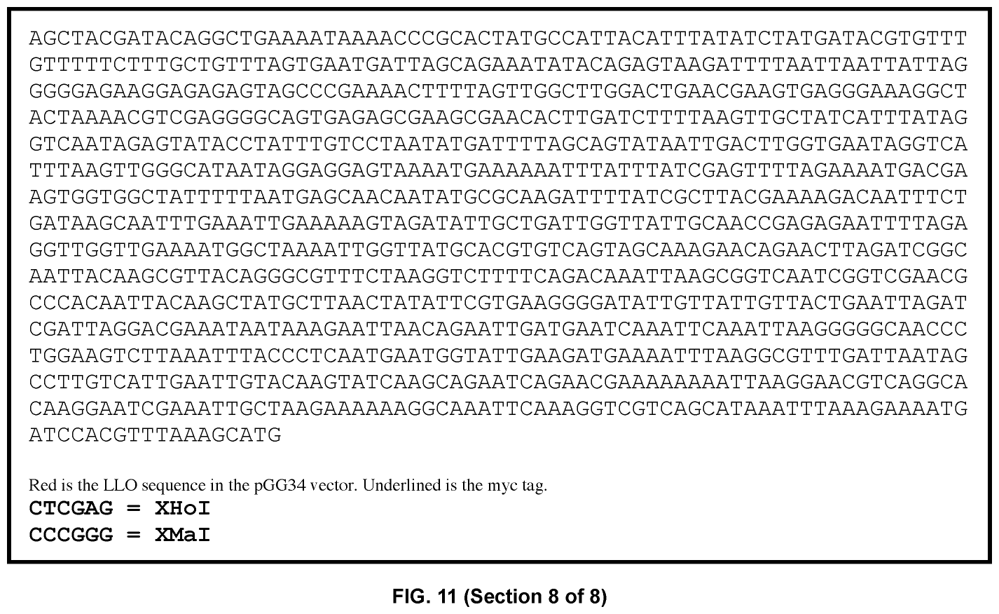

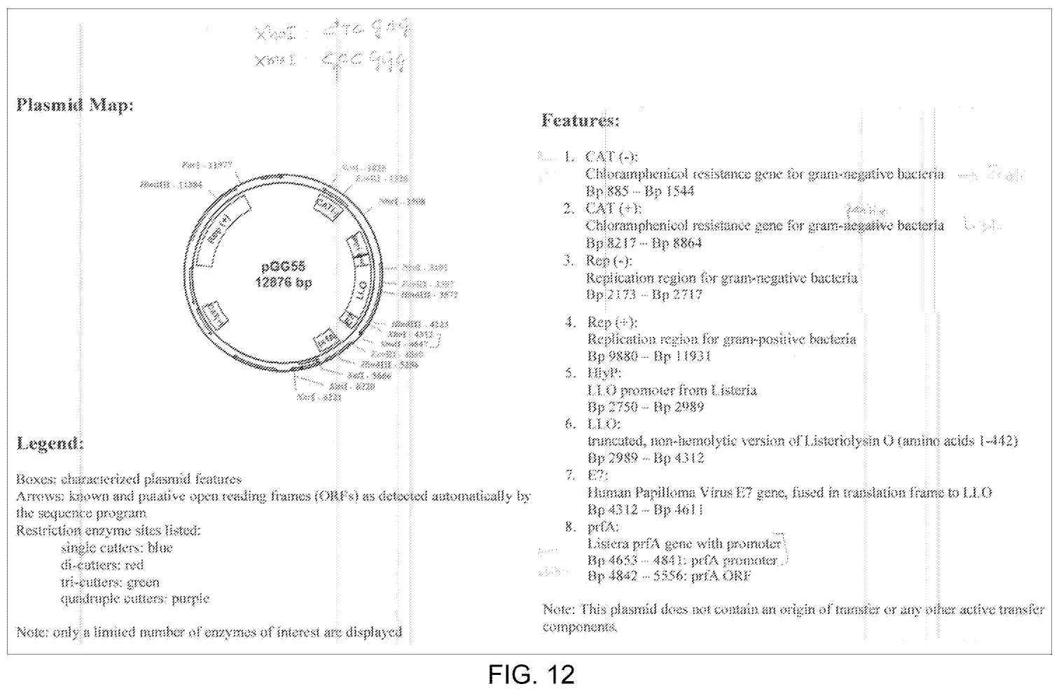

Attenuation of our Listeria consists of a Listeria background strain XFL-7 (see References 3P and 3V and FIG. 12) and a Listeria plasmid pGG34 (see FIG. 11). The XFL-7 lacks the prfA gene, which is required for survival. However, the plasmid does contain the prfA gene, but in a mutated format, which reduced the pathogenicity. Then the plasmid contains LLO in a truncated format (the C-terminal site that is responsible for binding cholesterol in the membranes, has been deleted). This reduces the pathogenicity, while maintaining Listeria's ability to escape the vacuole (most likely because the amino acid sequence PEST is still in the truncated LLO). In addition, the truncated LLO is also mutated. This further reduces the pathogenicity.

The tumor can be, for example, a tumor of one or more of the pancreas, ovary, uterus, neck, head, breast, prostate, liver, lung, kidney, neurones, glia, colon, testicle, or bladder. The tumor can be an inoperable tumor.

As used herein, “treating” a tumor means without limitation that one or more symptoms of the disease, such as the tumor itself, metastasis thereof, vascularization of the tumor, or other parameters by which the disease is characterized, are reduced, ameliorated, placed in a state of remission, or maintained in a state of remission. “Treating” a tumor also means without limitation that one or more hallmarks of the tumor may be eliminated or reduced by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries. Preferably, the method is effective to reduce tumor growth and/or size.

As used herein, reducing or preventing metastasis of a tumor means without limitation that any of the symptoms of the disease, such as the metastases, the extent of spread thereof, the vascularization of the metastases or other parameters by which the disease is characterized are reduced, ameliorated, prevented, placed in a state of remission, maintained in a state of remission, or eliminated. Preferably, the method is effective to reduce metastases. The method can reduce the incidence or likelihood of metastasis of a tumor.

As used herein, “protecting” against a virus means without limitation that infection of a subject by the virus or a variant of the virus is prevented to some degree or completely, or that if a subject is infected by the virus, one or more symptoms of the disease caused by the virus are reduced in amount, severity, or longevity.

As used herein, an mRNA-mediated virus vaccination antigen is an antigen to which a subject has previously been exposed through vaccination by an mRNA-mediated vaccine. Preferably, the antigen is an epitope of a protein a virus, the protein being a protein targeted by an mRNA-mediated vaccine against the virus.

Such antigens can include, for example, antigens used for vaccination against severe acute respiratory syndrome coronavirus 2 (SARS-CoV-2, also referred to as “coronavirus”), the virus that causes coronavirus disease 2019 (COVID-19). Many individuals have been or will be vaccinated and boosted with these antigens due to the COVID-19 pandemic, resulting in memory T cells (or other immune system components) that circulate in their blood stream for life or at least for an extended period of time. These memory T cells can be reactivated, even in a tumor microenvironment (TME). Examples of antigens that can be used include, but are not limited to, an epitope or a fragment containing one or more immunodominant epitopes of the SARS-CoV-2 virus. In a preferred embodiment, the antigen is a fragment of the spike protein of the SARS-CoV-2 virus containing one or more immunodominant epitopes.

### Virus Vaccines

More particularly, certain vaccines against old and new viruses have been shown to be effective, whether administered at a young age or an old age. Vaccines train the immune system to recognize the disease-causing part of a virus. Traditional vaccines contain either weakened viruses or purified signature proteins of viruses, and have enjoyed success in the effective elimination of certain viruses, especially when administered at a young age. These include without limitation vaccines for measles, mumps, rubella, tetanus, polio, smallpox, and influenza, among others. While such vaccines can also be effective when administered at an old age, the effectiveness is reduced in an older host (e.g., due to a lack of naive T cells, as described above).

Fortunately, newer, messenger RNA (mRNA) vaccines are effective even when administered at an old age. Rather than delivering a weakened virus or a purified signature protein of the virus, an mRNA vaccine delivers genetic material, mRNA, that encodes the viral protein fragment that is needed to train the immune system to recognize the virus. When the mRNA is injected, the cells at the injection site translate it to make the fragment directly in the body. Examples of such mRNA vaccines include the Pfizer-BioNTech COVID-19 Vaccine and the Moderna COVID-19 Vaccine, which have been designed to inoculate hosts against SARS-CoV-2. These vaccines are reported to be effective for approximately 95% and 94.1% of recipients, respectively, even in an older population.

While the invention will be described below primarily directed to use of the SARS-CoV-2 virus and the spike protein sequence of the SARS-CoV-2 virus, it should be understood that the invention encompasses use of any virus, and any effective protein sequence of the used virus.

### Directing Antibodies to Non-Conformational Epitopes to Limit or Eliminate Antibody-Mediated Immune System Escape Due to Variants and Other Virus Strains

While T cells will recognize 9-14 amino-acid length epitopes (CD4 and CD8) in the mRNA-mediated virus vaccination antigen protein, antibodies are often directed to conformational epitopes of antigens, but antibodies can also be directed to non-conformational epitopes. An advantage of directing antibodies to non-conformational epitopes is that these antibodies allow the immune system to better recognize a foreign microorganism and limit the organism's ability to escape the antibody-mediated immune system. This is because non-conformational epitopes are less likely to be sensitive to conformational changes in the target antigen. Conformational changes can occur in variants and other mutated versions of the original virus for which the vaccine is created against. Thus the approach of the present invention can in certain embodiments increase the ability of the antibody-mediated immune system to resist variants and other mutated copies of a virus that has been vaccinated against.

### Memory T Cell Production by mRNA-Mediated Virus Vaccination

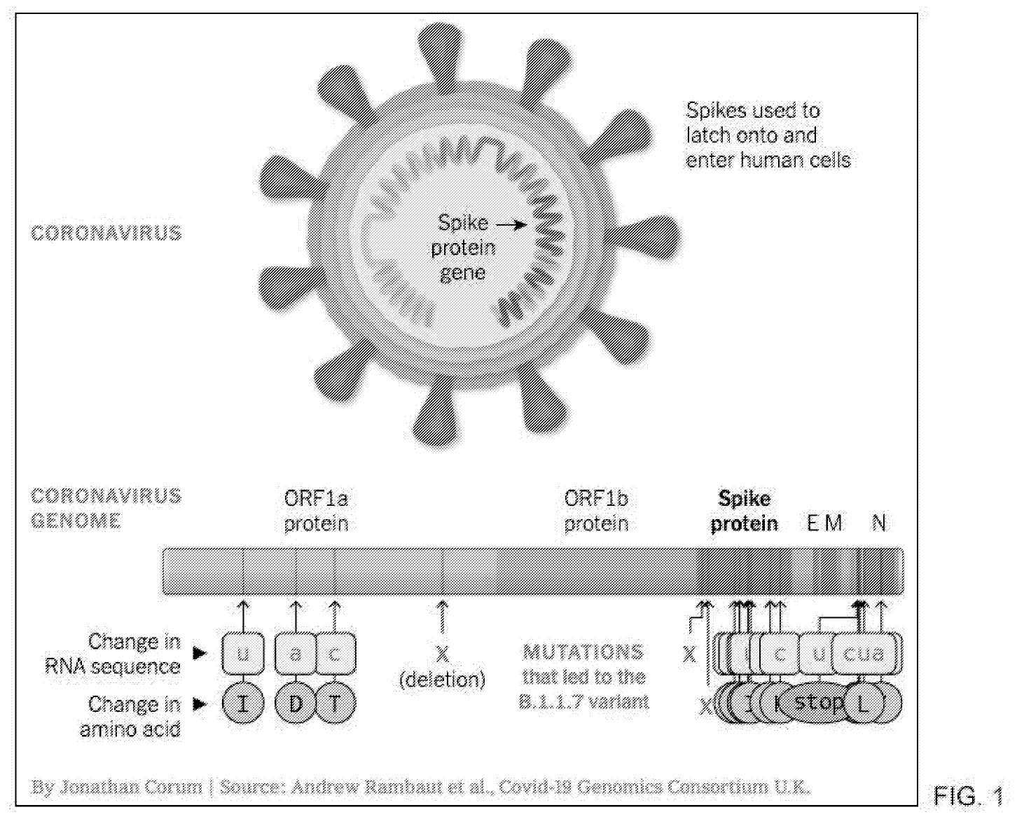

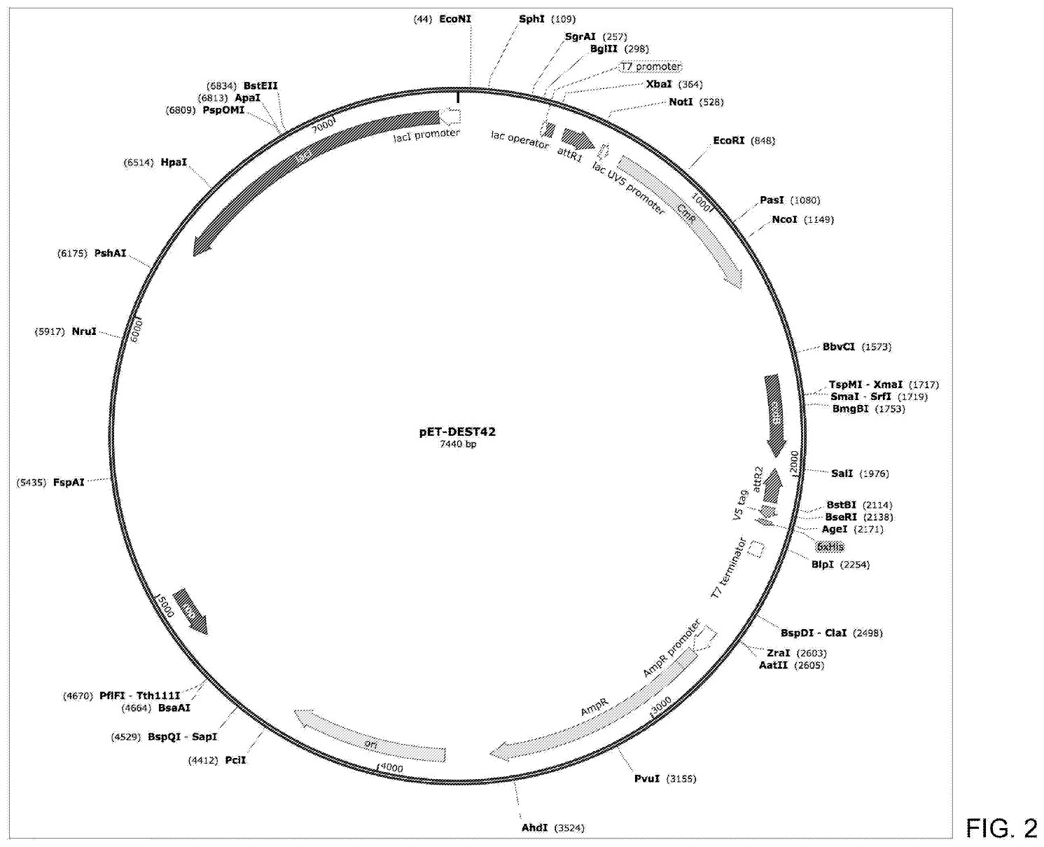

To address the COVID-19 pandemic, the SARS-CoV-2 genome was sequenced, and multiple vaccines were developed using the genetic information, including the Pfizer-BioNTech COVID-19 Vaccine and the Moderna COVID-19 Vaccine. The key protein sequence used for vaccination against SARS-CoV-2 is what is known as the “spike” protein of the virus, referred to herein as the “spike protein”, or simply “spike”. A visual representation of the SARS-CoV-2 genome, including the spike protein, can be found in FIG. 1.

The Spike glycoprotein (sometimes also called spike protein (see Reference 3X), is the largest of the four major structural proteins found in coronaviruses (see Reference 3Y). The function of the spike glycoprotein is to mediate viral entry into the host cell by first interacting with molecules on the exterior cell surface and then fusing the viral and cellular membranes. Spike glycoprotein is highly immunogenic. Antibodies against spike glycoprotein are found in patients recovered from SARS and COVID-19. Neutralizing antibodies target epitopes on the receptor-binding domain (See Reference 4A). Most COVID-19 vaccine development efforts in response to the COVID-19 pandemic aim to activate the immune system against the spike protein (see References 4B, 4C and 4D). (Source: https://en.wikipedia.org/wiki/Coronavirus_spike_protein.)

These vaccines have been administered to hundreds of millions of people, and will soon be administered to billions of people. It is estimated that once the vaccines are widely available, they will be administered to a significant percentage of the world's population. (Most of the world will need to receive a SARS-CoV-2 vaccine to achieve immunity, through vaccination or exposure, sufficient for the COVID-19 pandemic to subside.) Each recipient for which the vaccine is effective will generate an immune system memory of the spike protein and accordingly be vaccinated against it. If the vaccine is at least 94% effective as reported, then at least 94% of the recipients, even recipients of advanced age, will have sufficient production of memory T cells (and/or other immune system components) targeting the spike protein to kill the SARS-CoV-2 virus.

### Treating Cancer Using Vaccine-Generated or Infection-Generated Memory T Cells

In turn, in each person for which a SARS-CoV-2 vaccine is effective, the memory T cells (and/or other immune system components) targeting the spike protein can be used to effectively treat cancer according to the present invention. It should be understood that even if a vaccine does not provide long-term immunity, the treatment of the present invention can be used during the window of time in which the vaccine-provided immunity is effective. For example, if a vaccine provides 3 months of immunity, the method of the present invention can be used during those 3 months. Further, for example, if the immunity is boosted by additional vaccine administration, the method of the present invention can be used during the boost period as well.

Further, it should be understood that the present invention may be used to effectively treat cancer in persons who have contracted the SARS-CoV-2 virus and recovered, inasmuch as such persons may have naturally generated memory T cells (and/or other immune system components) targeting the spike protein (or another protein of the virus; for example, another protein which a naturally immune person's immune system has targeted), and such memory T cells (and/or other immune system components) can be used to effectively treat cancer according to the present invention.

### Determining Target Protein Sequence

For clarity, it should be understood that the present invention encompasses determining, from a naturally immune person, which protein sequence of a virus the person's immune system has targeted, developing a construct of the present invention that includes the determined protein sequence, and using the construct in accordance with the present invention to treat cancer in the person and/or provide protection from the virus in the person. For example, the sequence of the epitopes to which the T cells of the person's immune system are generated can be cloned into Listeria. For example, the epitopes can be determined by testing in the person reactions of antibodies to different fragments of the virus. And, for example, it is possible to use pseudo antibodies (i.e., antibodies against other antibodies), which through vaccination and subsequent sequencing, allow examination of potential epitopes.

For further clarity, it should be understood that the present invention encompasses using in the construct(s) (1) a protein of the virus that vaccine manufacturers decided to use as a target for a vaccine, (2) a portion of the protein of the virus that vaccine manufacturers decided to use as a target for a vaccine (e.g., a portion independently determined to be most effective), and/or (3) a different protein of the virus altogether, as indicated above or otherwise.

### As Alternative or Supplemental to Traditional Cancer Treatments

Traditional cancer treatments, such as chemotherapy or other uses of chemotherapeutic agents, can weaken a person's immune system. Because the present invention does not pose such a danger, it can be used instead of or in addition to such traditional cancer treatments.

It should be understood that the present invention can be used to treat even cancers for which there are practically no effective treatments, such as pancreatic cancer, which is almost always detected in metastatic form, or ovarian cancer. Good candidates also include cancers for which surgery to remove the primary tumor is often not an option because of tumor location, such as head and neck cancers or inoperable hepatocellular carcinoma. A third cohort of patients that would be expected to benefit from the present invention are patients with various types of metastatic disease, which is recurrent or refractory to standard treatments, such as, for example, lung and colon cancers, as well as breast cancer.

### Ovarian Cancer Example

As an example, ovarian cancer is prone to recurrence because of the development of cancer stem cells (CSCs) with a capacity of unlimited self-renewal and proliferative capacity (See Reference 3F) and resistance to chemotherapy (See Reference 3E). It has been reported that these CSCs account for the development of a chemoresistant population of tumor cells disseminated throughout the peritoneal cavity which cannot be eliminated by traditional treatment modalities such as surgery or radiation and only very modestly respond to targeted therapies.

In addition, due to the ongoing COVID-19 pandemic, many cancer patients are experiencing delayed treatment. Cancer patients, who are more likely to be elderly, are amongst the most vulnerable to COVID-19, because their immune system is less effective than those of young adults, due to age-related impairments of the immune system (See References 3G, 3H, 3I, 3J and 3K).

### Mechanism of a Preferred Embodiment of the Present Invention

To address these issues, the dual strategy of the present invention was developed and is disclosed herein, for targeting both cancer (e.g., chemoresistant cancer) and a virus (e.g., the SARS-CoV-2 virus), using an attenuated non-toxic and non-pathogenic bacterium Listeria monocytogenes expressing a protein of the virus (e.g., the spike protein of the SARS-CoV-2 virus).

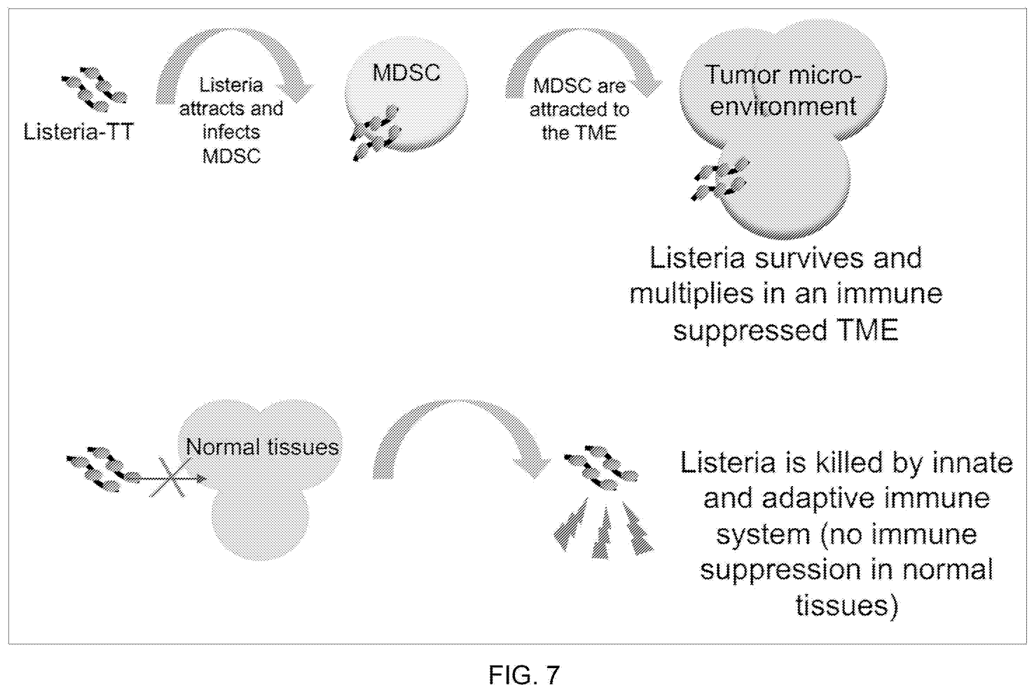

The disclosed unique bacterium selectively delivers highly immunogenic antigens (e.g., the spike protein) into tumor cells through the following cascade of events: (i) Listeria attracts and infects myeloid-derived suppressor cells (MDSCs), (ii) the infected MDSCs are attracted to the tumor microenvironment (TME), and (iii) Listeria spreads from the MDSCs into tumor cells (see FIG. 7).

Due to prior infection or inoculation, a large portion of the current worldwide population has memory T and B cells against the spike protein. Reactivation of these pre-existing spike-specific memory T cells by the Listeria-Spike construct of the invention will result in killing of the infected tumor cells through the standard memory T-cell mediated immune system pathways, while the spike-specific B cells will produce the antibodies to the spike protein. These antibodies will then prevent the infection of cells with the SARS-CoV-2 virus. According to the invention, the spike-specific T cells will kill both chemoresistant and non-resistant tumor cells since they target the spike protein and do not use pathways involved in proliferation or oncogenesis.

The selective delivery of the spike protein inside tumor cells is possible because Listeria survives and multiplies in the tumor microenvironment (TME) as a result of strong immune suppression, but is rapidly eliminated by the immune system in normal tissues that lack immune suppression. It has been shown that this mechanism is successful using the recall antigen tetanus toxoid in mice with pancreatic and ovarian cancer (see Reference 3W). According to the present invention, Listeria expressing the spike protein targets both (1) cancer cells infected with the Listeria-Spike construct and (2) the SARS-CoV-2 virus, which contains the spike protein.

Therefore, the present invention in preferred embodiments provides a pharmaceutical composition, therapeutic compound and/or vaccine that both (simultaneously or separately) treats cancer and protects against a virus. The present invention in preferred embodiments further provides a method of developing the same.

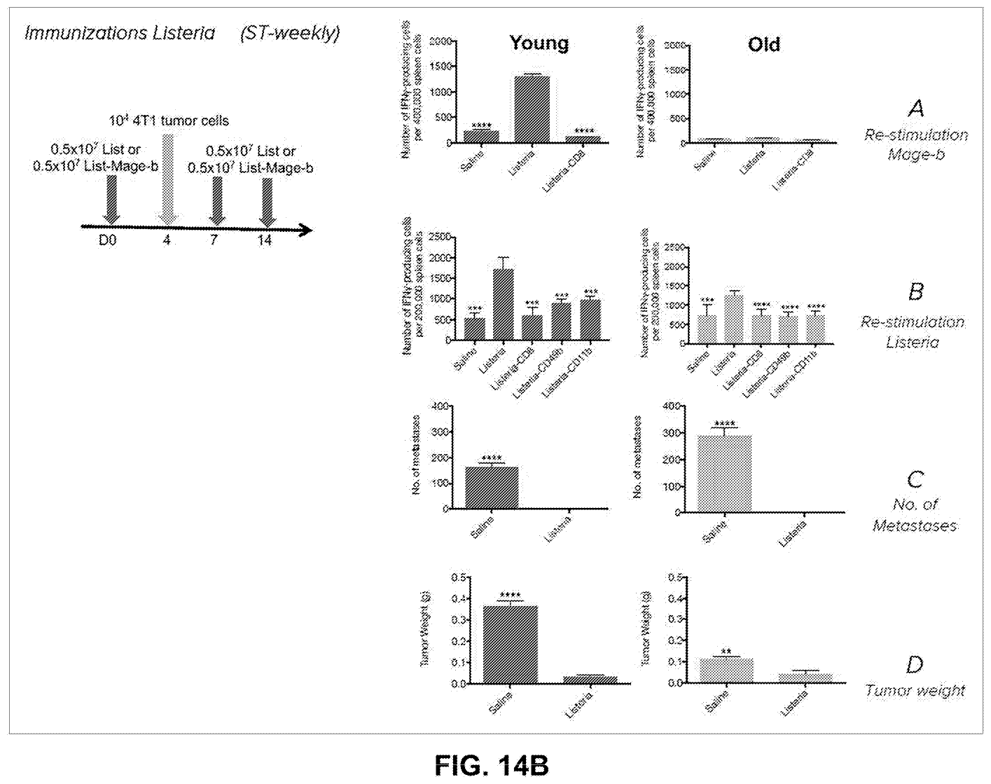

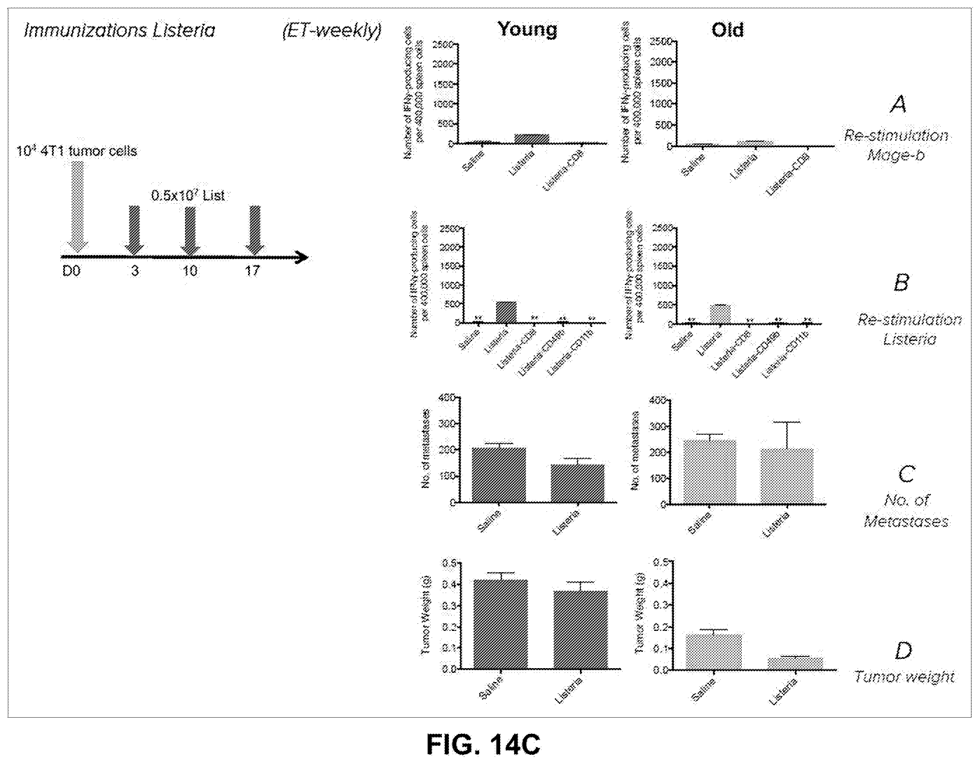

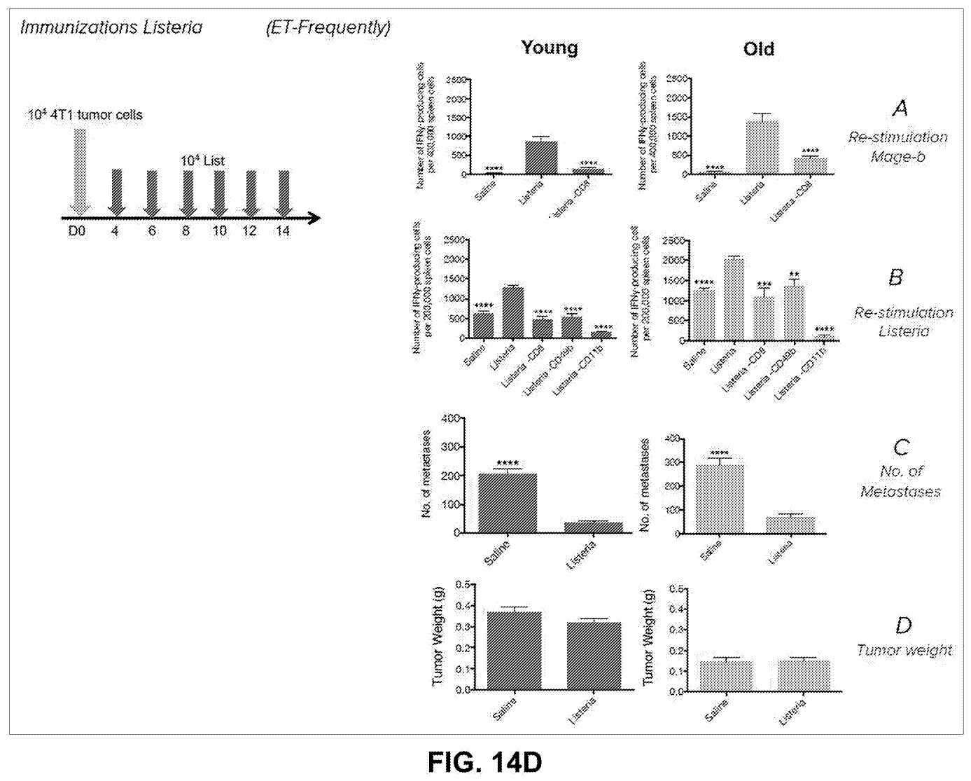

According to the present invention, multiple low doses of a Listeria-protein construct are superior in generating T cell responses to a target protein compared to three high doses, resulting in a significantly stronger effect on metastases, at old and young ages (see FIGS. 14A-D). Therefore, according to the present invention, high doses of a Listeria-Spike construct are useful to deliver the spike protein into tumor cells, and low doses of a Listeria-Spike construct are useful to to reactivate T cells (and B cells) to the spike protein.

According to a preferred embodiment of the present invention, a method of developing a pharmaceutical composition, therapeutic compound and/or vaccine that both (simultaneously or separately) treats cancer and protects against a virus, includes one or more of the following steps.

### Step 1: Generation and Characterization of Listeria-Spike

This step addresses at least two issues. Recurrent ovarian cancer is extremely difficult to treat and cancer patients, who are typically old, are the most vulnerable to the SARS-CoV-2 virus and react less efficiently to virus vaccines than do young adults. Therefore, in this step the spike protein of the SARS-CoV-2 virus is cloned into Listeria as an antigen (most individuals are immunized against SARS-CoV-2 as discussed above, or will be, or can be) that now functions as an alternative for neoantigens (e.g., this is important because ovarian cancer and many other cancers poorly express neoantigens, making them a bad target for neoantigen based therapeutic vaccines) and as an antigen to prevent infection by the SARS-CoV-2 virus and/or or avoid or reduce symptoms of COVID-19, particularly in old age (but also at young ages).

### Step 1a. Cloning the Spike Protein DNA Sequence Into Listeria

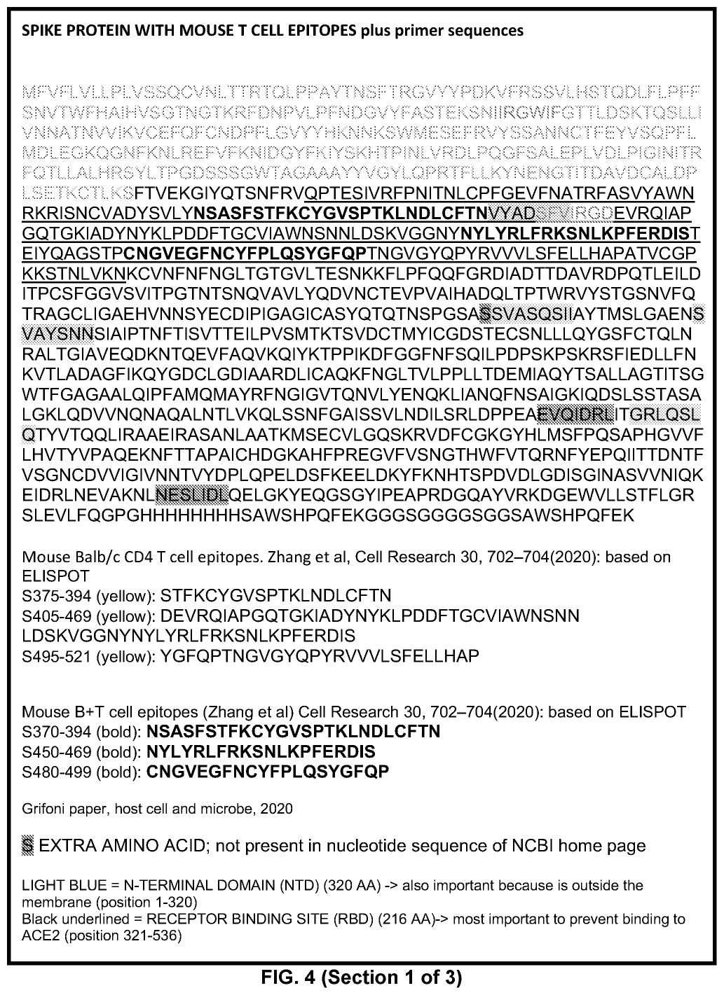

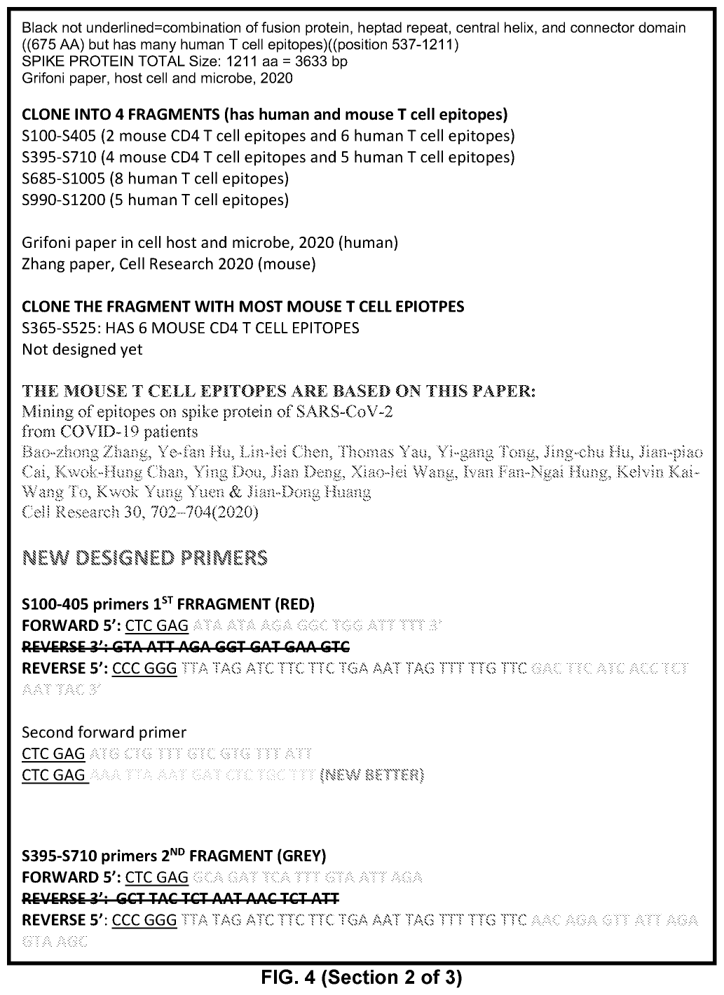

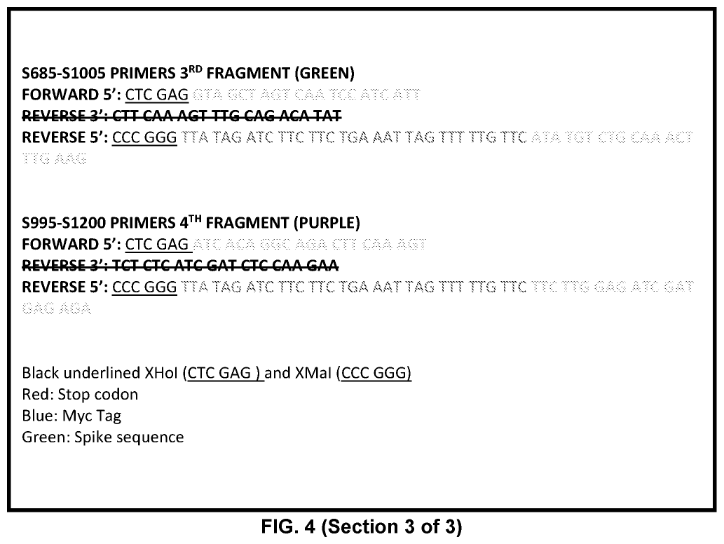

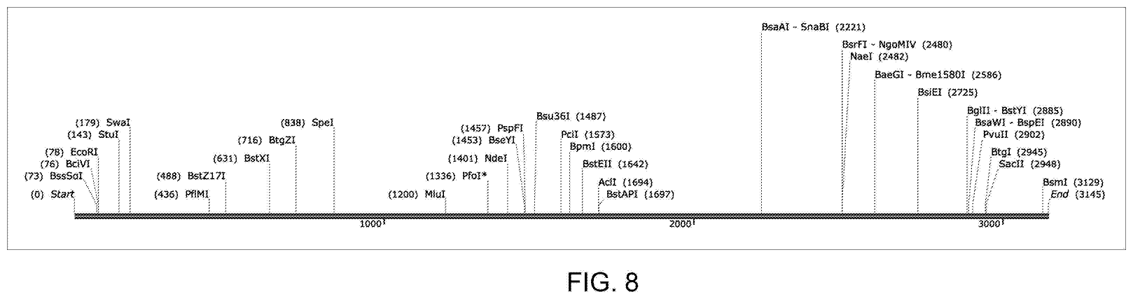

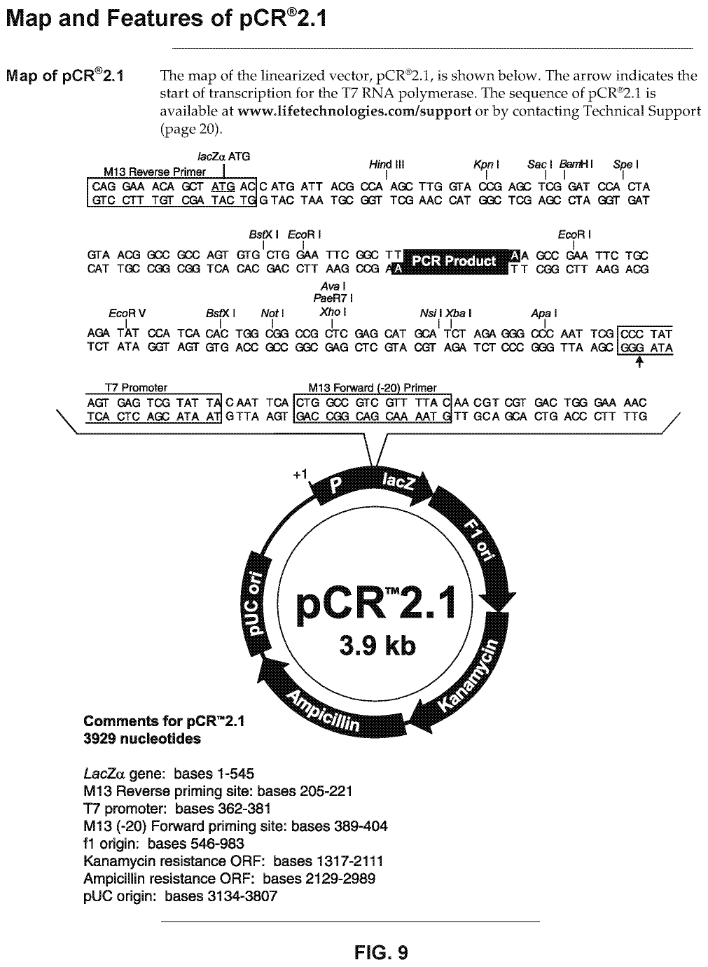

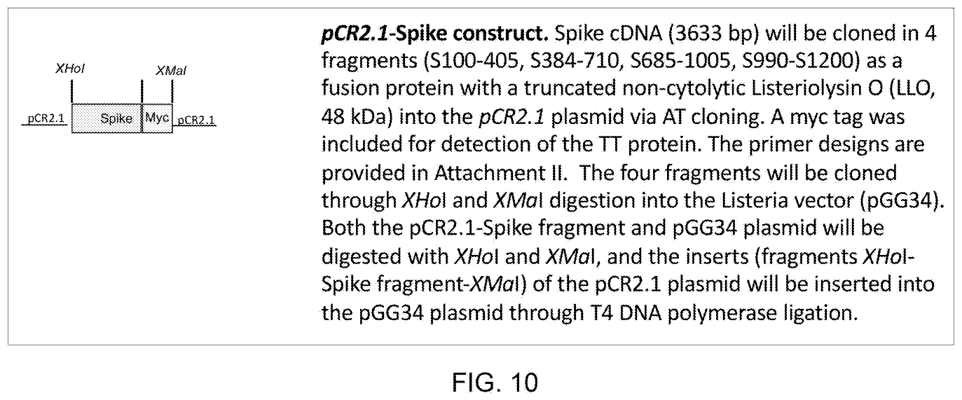

The spike protein DNA sequence was obtained and primers were developed that fuse the spike protein with truncated Listeriolysin-O (LLO) in the Listeria vector. Since the spike protein size (e.g., 3582 nucleotides) is larger than the carrying capacity of the Listeria, the spike protein was cloned in four fragments into the Listeria vector using the primers shown in FIG. 4. Briefly, Listeria-Spike is cloned into pCR2.1 (see FIG. 9) using primers containing XHoI and XMaI restriction sites and a myc Tag for detection of spike using the primers of FIG. 4. XHoI and XmaI restriction sites were selected because they are not present in the spike protein (see FIG. 8). Subsequently the pCR2.1-XHoI-Spike-Myc-XmaI (see FIG. 10) and pGG34 plasmids (see FIG. 11) are digested with XHoI and XMaI, and the XHoI-Spike-myc-XMaI DNA fragment is cloned into the pGG34 vector, and then electroporated into the Listeria background strain XFL7 (See References 3P and 3V). Preferably, since new variants of SARS-CoV-2 (and accordingly new variants of the corresponding spike protein) may develop continuously, the latest spike protein variant is cloned into the Listeria.

### Step 1b. Characterization of Listeria-Spike

This step includes DNA sequencing, western blotting, and functional assays demonstrating that Listeria-Spike is able to infect and kill tumor cells. To detect the expression and secretion of the spike protein, antibodies to Myc Tag (which is commercially available) are preferably used, and LLO is detected by anti-LLO antibodies. Preferably, tumor cell lines Id8-Luc and p53^{-/-}; Ccne1^{OE}; Akt2^{OE}; Kras^{OE} are used.

According to the present invention, the spike protein functions as an antigen and strong T and B cell responses against the spike protein contribute to the treatment (e.g., reduction/elimination) of cancer cells and to the protection from the virus (e.g., SARS-CoV-2). Multiple human and mouse T and B cell epitopes in the spike protein have been published. Accordingly, the Listeria-Spike construct can be used in clinical trials.

According to the present invention, T cells will recognize 9-14 as epitopes (CD4 and CD8) in the four cloned fragments of the spike protein. Inasmuch as this may affect the antibody production in preventing the infection of cells, the present invention encompasses analyzing the antibody production and adjusting the Listeria-Spike construct to address any non-optimal results. For example, antibodies are often directed to conformational epitopes like the spike protein (See Reference 3Q), but antibodies can also be directed to non-conformational epitopes. It is anticipated that in certain embodiments, antibodies to non-conformational epitopes may provide an advantage because such epitopes often limit the microorganism to escape the antibody-mediated immune system (See References 3R and 3S).

### Step 2: Testing Listeria-Spike in Various Mouse Models

This step evaluates whether Listeria-Spike is effective against a cancer (e.g., ovarian cancer). Preferably, two mouse tumor models are selected.

As an example, the first mouse model Id8 expresses Luciferase and is useful for determining the effect of Listeria-Spike against mouse ovarian cancer not only at the end but also during therapy and after therapy (measuring durable effects). Although clinically this HGSC model also develops tumor nodules all over the peritoneal cavity like in humans, it lacks any of the frequent mutations in HGSC (See Reference 3T). To come closer to the human ovarian cancer, the Id8-Luc with a deletion in the Trp gene (Trp53-/-) was used.

As another example, the second mouse model, the p53^{-/-}; Ccne1^{OE}; Akt2^{OE}; Kras^{OE} mouse model (See Reference 3U), is genetically much closer to human ovarian cancer and also develops multiple tumor nodules in the peritoneal cavity, but does not express luciferase and will be analyzed by determining the number of nodules by the naked eye.

Id8-luciferase mouse model: Using this model, efficacy and survival studies are performed in old mice with cancer. This model develops high grade serous ovarian carcinoma upon injection of the Id8 tumor cells into the peritoneal cavity within 3 weeks, with severe ascites production.

p53^{-/-}; Ccne1^{OE}; Akt2^{OE}; Kras^{OE} mouse model: Using this model, efficacy and survival studies are performed in old mice with cancer. This latter mouse model is genetically closer to human ovarian cancer.

Preferably, in both studies, C57BL/6 mice of 20 months are used. According to the present invention, because multiple low doses of Listeria were determined to be superior in generating T cell responses to a target protein compared to three high doses resulting in a stronger effect on metastases, at young and at old age (see FIGS. 14A-D), the method of the invention preferably uses high doses to deliver the spike protein into the tumor cells, and multiple low doses to reactivate the T cells (and B cells) to the spike protein.

This step further includes determining through which mechanism(s) T cells kill tumor cells. Preferably, tumor tissues sections of treated and control mice are analyzed by Nanostring nCounter system. This system enables the counting of hundreds of RNA, DNA, or protein targets using single-molecule imaging of molecular “barcodes”, enabling digital quantitation of hundreds of unique targets in a single reaction. Such a technology delivers robust data on all samples, including formalin-fixed paraffin-embedded (FFPE) tissue.

### Step 3: Testing Listeria-Spike-Generated B and T Cells Against SARS-CoV-2 Infection In Vitro

### Step 3a Testing Antibodies

This substep addresses testing antibodies to prevent SARS-CoV-2 infection. To test whether antibodies of Listeria-Spike-vaccinated mice protect against SARS-CoV-2 infection, neutralization assays with a recombinant vesicular stomatitis virus carrying SARS-CoV-2 S protein (rVSV-SARS2) are used.

### Step 3. Use of T Cells

This substep addresses testing T cells to prevent SARS-CoV-2 infection. A similar assay will be used but now instead are added T cells of Listeria-Spike-vaccinated mice to the assay. According to the present invention, the T cells eliminate SARS-CoV-2-infected cells.

### Generation and Characterization of Listeria-Spike

As will be explained in greater detail below, the invention encompasses a Listeria monocytogenes-mRNA-Mediated Vaccination Virus Antigen (Lm-mMVVA) construct, a method of developing the same, and a method of use thereof to treat cancer and/or provide protection against a virus and/or its effects.

Further with regard to Step 1 (Generation and Characterization of Listeria-Spike) above, a preferred example of a Lm-mMVVA construct is a Listeria monocytogenes-SARS-CoV-2 spike protein construct (referred to herein as “Listeria-Spike” or “Lm-SP”), as described herein (see FIG. 4).

As indicated above and illustrated in FIG. 1, the genome of the SARS-CoV-2 virus has been sequenced, including the sequence of the spike protein. Reference 1A, Reference 1B, and FIG. 2 discuss and illustrate a vector (pET-DEST42 plasmid) (see FIG. 2) into which the spike protein is cloned and then from which the spike protein is purified, according to the present invention. References 1C, 1D and 4E provide the spike protein sequence.

The spike proteins of SARS-CoV-2 are formed on the surface of the virus and are a key component of the cellular infection machinery used by the virus. Accordingly, according to the present invention, the spike protein can be used as an antigen for targeting by the host's immune system. That is, the spike protein evokes an antibody response in the host, such that memory T cells (and/or other immune system components) are generated to bind to the epitope of the antigen and recognize and destroy the virus. Accordingly, a key to fighting the SARS-CoV-2 virus is determining the epitopes of the spike protein that most effectively evoke an immune system response in the host.

The epitopes of the spike protein to which antibodies in mice will bind may be different from the epitopes of the spike protein to which antibodies in humans will bind. There are many human T cell epitopes and many mouse T cell epitopes present in the spike protein sequence. Therefore, in accordance with the present invention, the spike protein of the virus was studied and human T cell epitopes and mouse T cell epitopes, as well as human B cell epitopes and mouse B cell epitopes, were identified. While any method of identification is encompassed by the present invention, the human T cell epitopes can be, and were, identified by algorithms, and the mouse T cell epitopes can be, and were, identified by enzyme-linked immunospot (ELISpot) analysis. Reference 1E and Reference 1F discuss methods of identifying epitopes.

Preferably, the Lm-SP construct includes the T cell epitope(s) and B cell epitope(s) that evoke the most effective immune response. Accordingly, preferably, according to the present invention, to determine the most effective T cell epitopes to include, an initial Lm-SP construct is prepared with all of the identified T cell epitopes and B cell epitopes included.

Further preferably, according to the present invention, accordingly if necessary, to account for a possibility that not all of the T cell epitopes and B cell epitopes were identified, the initial Lm-SP construct is prepared with the entire spike protein sequence included. For example, it may be that there are more T cell epitopes and B cell epitopes in the spike protein than have been identified. Reference 1G describes the number of human T cell epitopes and mouse T cell epitopes present in the spike protein, but only with regard to a fraction of the entire spike protein sequence. Reference 1H further provides human T cell epitopes and mouse T cell epitopes present in the spike protein.

Further, preferably, according to the present invention, both human T cell epitopes and mouse T cell epitopes (and human B cell epitopes and mouse B cell epitopes) are included together in the Lm-SP construct, so that the construct can be used for both human and mouse experimentation without modification (e.g., in the case that results from a trial of a construct in mice support use of the construct in human trials).

In certain embodiments of the present invention, it may be necessary or desirable to prepare multiple constructs. For example, depending on the size of the antigen sequence versus the Listeria payload size limit (or depending additionally or alternatively on other factors), the antigen sequence can be separated into subsequences, each subsequence being included in a respective construct.

For example, the separation point(s) of the sequence, the sizes of the subsequences, overlap (if any) of the subsequences, and what amino acids to include in or exclude from the subsequences, can according to the present invention be based on epitope analyses and/or other analyses to select the highest immunogenicity and/or other properties that are advantageous or otherwise desirable. Without limitation, any valuable factor can be considered.

Separation of the sequence into subsequences according to the present invention can be accomplished such that the entire sequence is represented as multiple contiguous subsequences in multiple constructs. For example, if the entire spike protein sequence is too large to fit into a single construct, multiple constructs can be prepared, each one including a respective portion of the spike protein sequence, such that together the portions encompass the entire spike protein sequence. (It should be understood that the respective portions can include part or all of one or more of the other portions.) For example, the spike protein sequence is too large to clone in its entirety into Listeria for the purposes of certain embodiments of the present invention. The spike protein sequence contains 3,582 nucleotides, whereas the maximum number of nucleotides that can be cloned into Listeria and maintain effectiveness of the construct is approximately 1,024 nucleotides, because the cloned sequence must be secreted and the secretion process is less efficient for larger proteins (e.g., proteins having greater than approximately 1,024 nucleotides).

Accordingly, preferably one or more constructs are prepared, together covering all T cell epitopes and B cell epitopes of the spike protein. Further preferably, all human T cell epitopes and B cell epitopes of the spike protein and all mouse T cell epitopes and B cell epitopes of the spike protein are covered. Still further preferably, the entire spike protein sequence is covered.

In the present example according to the present invention, four constructs were prepared, together covering the entire spike protein sequence, therefore accordingly covering all human T cell epitopes and B cell epitopes of the spike protein and all mouse T cell epitopes and B cell epitopes of the spike protein, and therefore accordingly covering all T cell epitopes and B cell epitopes of the spike protein.

With regard to the preparation of the constructs in accordance with the present invention, antibodies to Listeriolysin-O (LLO) were used to select the assembled constructs because the spike protein sequence was cloned as a fusion protein with LLO.

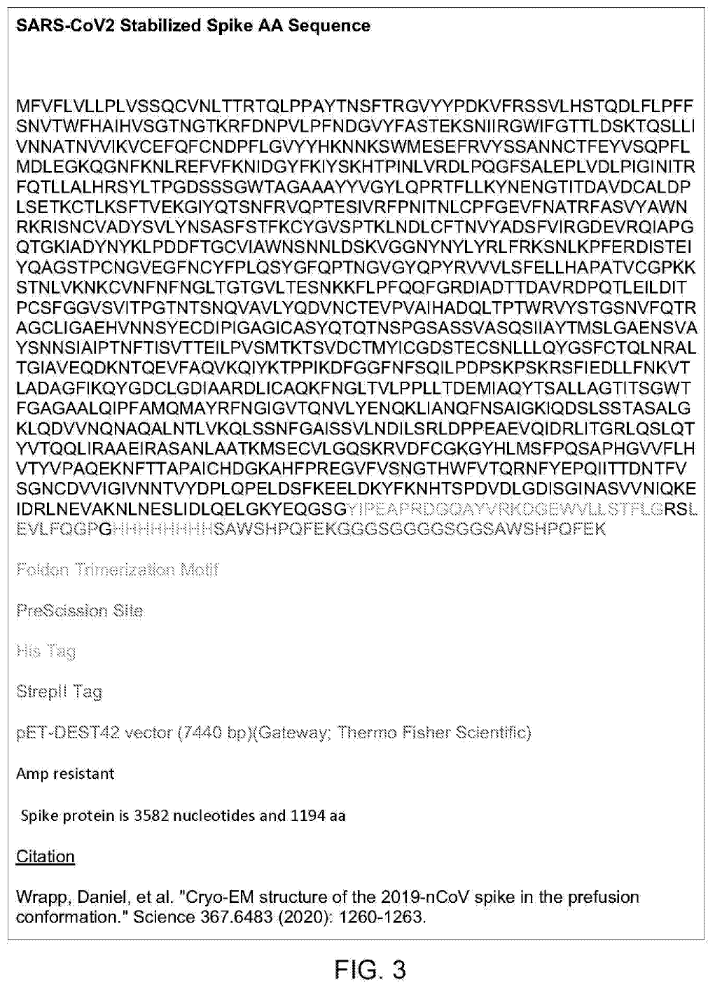

Further with regard to the preparation of the constructs in accordance with the present invention, the spike protein sequence was combined with a pET-DEST42 plasmid to facilitate manufacture of the spike sequence payload through polymerase chain reaction (PCR) and direct cloning into Listeria. FIG. 2 illustrates the pET-DEST42 plasmid. FIG. 3 illustrates the spike protein sequence ready for insertion into the pDEST42 plasmid.

Still further with regard to the preparation of the constructs in accordance with the present invention, in a survey for restriction sites it was determined that XHoI and XMaI sites are not present in the spike protein sequence. Accordingly, these two restriction enzymes were used for the cloning. It should be understood that other restriction enzymes can be used for the cloning, so long as their associated sites are not present in the spike protein.

FIG. 4 describes the identified mouse T cell epitopes of the spike protein in detail. The mouse T cell epitopes and primers of each construct are marked in the presented amino acid sequence. The primers for each of the four constructs include an myc tag to detect the spike protein sequence. It should be understood that additionally or alternatively, other tags can be used to enable or allow identification, selection, and purification of sequences that have the tags attached to the end.

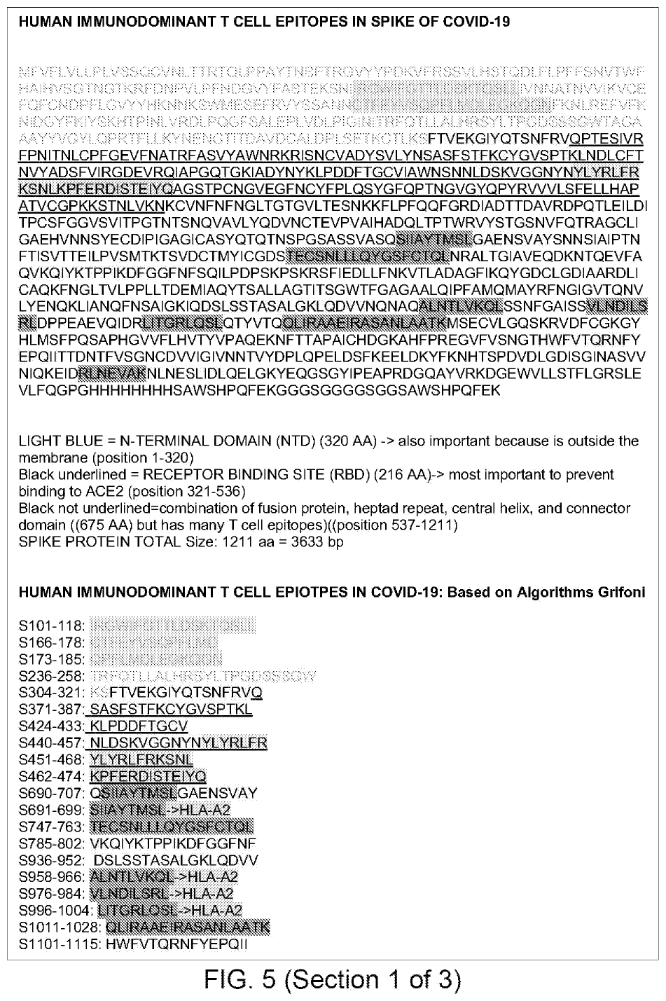

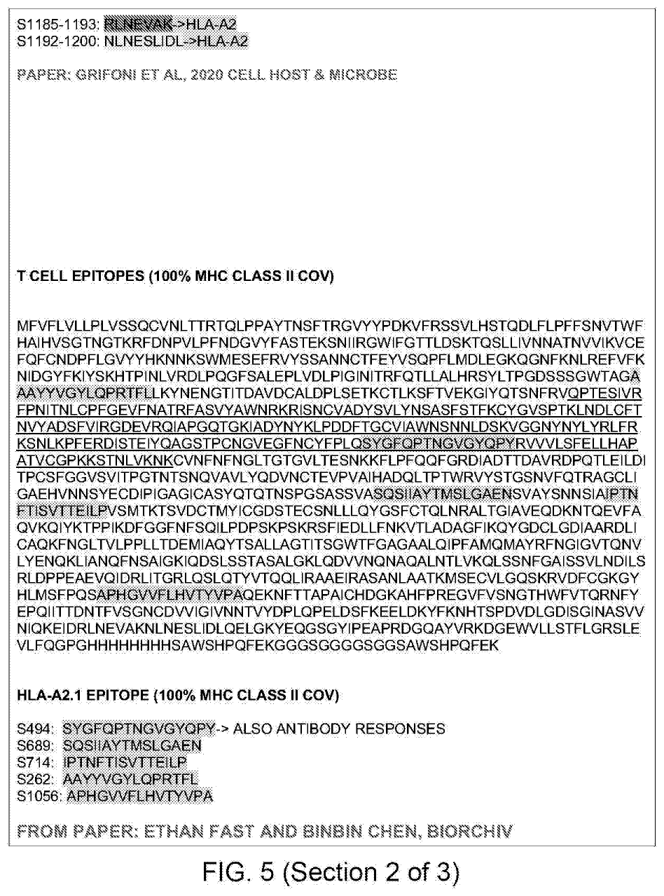

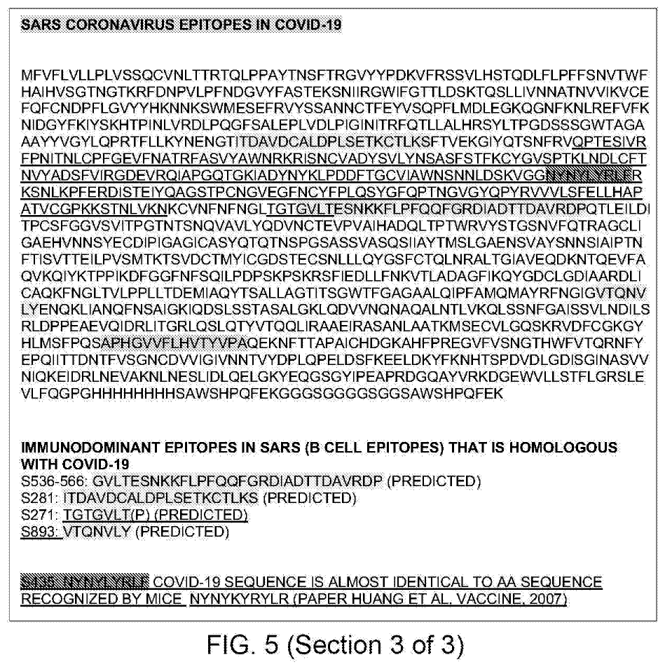

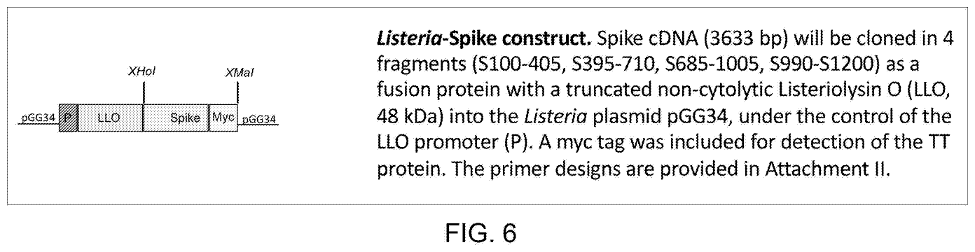

FIG. 5 describes the identified human T cell epitopes of the spike protein in detail. The human T cell epitopes and primers of each construct are marked in the presented amino acid sequence. The primers for each of the four constructs include an myc tag to detect the spike protein sequence. It should be understood that additionally or alternatively, other tags can be used to enable or allow identification, selection, and purification of sequences that have the tags attached to the end.

It is preferable, in testing the constructs, to select first the construct having the subsequence with the potentially most desirable characteristics. For example, it is preferable to obtain the best reaction possible in preclinical experiments in mice while also maximizing the potential for treatment success in humans. That is, regulatory bodies (e.g., the U.S. Food and Drug Administration) may require promising results in mice before permitting human trials, and further may require that there be no change to the tested construct between the testing in mice and the testing in humans. Therefore, a construct that contains the most human and mouse epitopes is preferred for initial testing.

Accordingly, preferably, the most promising construct to test, of the constructs in FIGS. 4 and 5, is the construct that includes the most mouse T cell epitopes and the most human T cell epitopes, which is the second of the four constructs.

Preferably, once a construct shows effectiveness in mice, the same construct is tested in a limited human trial. Further preferably, if the limited human trial demonstrates safety, the construct can be tested in expanded human trials. If a construct causes negative results, other constructs can be tested. Of course, multiple constructs can be tested simultaneously in multiple trials. Once a construct is shown to be effective in human trials, experimentation can be undertaken to determine which T cell epitope of the construct is most effective, with a goal being to create a construct that includes only the most effective T cell epitope, so as to minimize side effects and/or other negative outcomes.

With regard to use of the constructs to treat cancer, preferably, prior to administration to the subject, the bacteria are cultured in a yeast medium. The method can further comprise administering Cytosine-phosphate-Guanine (CpG) to the subject as an adjuvant.

In one embodiment, prior to administration of bacteria to the subject, the subject is screened for their major histocompatibility complex (MHC) 1 haplotype and administered an mRNA-mediated virus vaccination antigen for which the subject shows a CD8 T cell response.

Bacteria can be administered by different routes to the subject. For example, bacteria can be administered systemically to the subject, such as for example, by intravenous administration. Bacteria can be administered by direct injection to a tumor site in the subject. Myeloid-derived suppressor cells (MDSCs) can be used to deliver attenuated bacteria to the microenvironment of both primary and metastatic neoplastic lesions, where the attenuated bacteria spread from MDSCs into tumor cells (see, e.g., Reference 2J). The infected tumor cells then become a target for activated immune cells.

Preferably, the subject receives repeated administrations of attenuated bacteria that expresses the mRNA-mediated virus vaccination antigen. For example, the administration may be daily or every other day, for a period of several days until a satisfactory therapeutic outcome is achieved.

The method can further comprise administering to the subject a chemotherapeutic agent that reduces the number of myeloid-derived suppressor cells (MDSCs). Such chemotherapeutic agents include, for example, gemcitabine, Vitamin A derivates, Amiloride, CpG oligodeoxynucleotide (CpG ODN), Docetaxel, 5-Fluorouracil, GW2580, Sildenafi and Sinitinib (see, e.g., References 2C and 2D).

### Example Protocol for Treating Subjects with Listeria-Spike

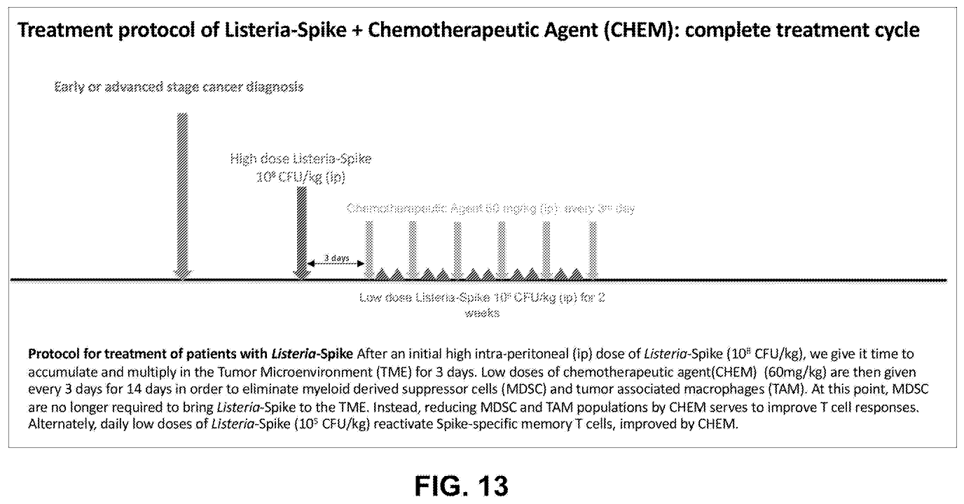

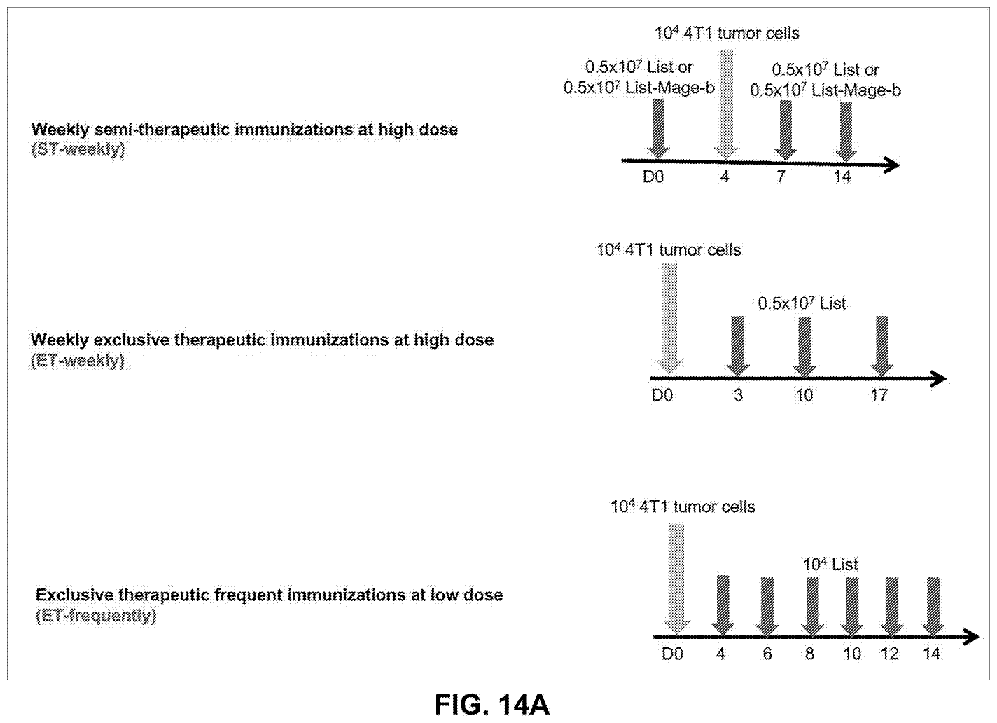

The following is an example of a protocol, according to the present invention, for treatment of subjects (e.g., patients) with Listeria-Spike. (See FIG. 13.)

After an initial high intra-peritoneal (ip) dose of Listeria-Spike (108 CFU/kg), time is allowed for it to accumulate and multiply in the Tumor Microenvironment (TME), preferably for 3 days. Low doses of chemotherapeutic agent (CHEM) (60 mg/kg) preferably are then given, preferably every 3 days for 14 days, in order to eliminate myeloid derived suppressor cells (MDSCs) and tumor associated macrophages (TAMs). At this point, MDSCs are no longer required to bring Listeria-Spike to the TME. Instead, reducing MDSC and TAM populations by CHEM serves to improve T cell responses. Alternately, low doses (preferably given daily) of Listeria-Spike (105 CFU/kg) reactivate Spike-specific memory T cells, improved by CHEM.

### Compounds, Compositions, Virus Vaccines and Cancer Vaccines

The present invention encompasses a cancer immunotherapy compound comprising attenuated bacteria that expresses an mRNA-mediated virus vaccination antigen. The bacteria can be, for example, one of more of Listeria monocytogenes, Salmonella thyphimurium, Vibrio cholera, Clostridium, and Bifidobacterium breve. The antigen can be, for example, an epitope of the spike protein of the SARS-CoV-2 virus.

The present invention further encompasses a pharmaceutical composition comprising a pharmaceutically acceptable carrier and attenuated bacteria that expresses an mRNA-mediated virus vaccination antigen. The bacteria can be, for example, one of more of Listeria monocytogenes, Salmonella thyphimurium, Vibrio cholera, Clostridium, and Bifidobacterium breve. The antigen can be, for example, an epitope of the spike protein of the SARS-CoV-2 virus.

Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. The pharmaceutically acceptable carrier used can depend on the route of administration. The pharmaceutical composition can be formulated for administration by any method known in the art, including but not limited to, oral administration, parenteral administration, intravenous administration, transdermal administration, intramuscular administration, intranasal administration, direct injection into a tumor site, and administration through an osmotic mini-pump.

The present invention further encompasses a virus vaccine comprising attenuated bacteria that expresses an mRNA-mediated virus vaccination antigen. The bacteria can be, for example, one of more of Listeria monocytogenes, Salmonella thyphimurium, Vibrio cholera, Clostridium, and Bifidobacterium breve. The antigen can be, for example, an epitope of the spike protein of the SARS-CoV-2 virus.

The present invention further encompasses a therapeutic cancer vaccine comprising attenuated bacteria that expresses an mRNA-mediated virus vaccination antigen. The bacteria can be, for example, one of more of Listeria monocytogenes, Salmonella thyphimurium, Vibrio cholera, Clostridium, and Bifidobacterium breve. The antigen can be, for example, an epitope of the spike protein of the SARS-CoV-2 virus.

The approach of the present invention overcomes the above discussed problem of poorly immunogenic antigens in cancer vaccination by using immunotherapy compounds with highly immunogenic mRNA-mediated virus vaccination antigens. The procedure involves reactivating memory T cells to foreign highly immunogenic antigens to which individuals have been previously exposed through vaccination by an mRNA-mediated vaccine (or by infection by the virus itself), and by the selective delivery of these antigens into tumor cells by an attenuated non-toxic and non-pathogenic bacterium, such as Listeria monocytogenes. These memory T cells will kill infected tumor cells presenting the highly immunogenic antigens. In previous studies, Listeria has been used for the selective delivery of anticancer agents to the tumor microenvironment and into tumor cells of metastases and tumors. Listeria was effectively cleared by the immune system in normal tissue but not in the heavily immune-suppressed microenvironment of metastasis and primary tumor (see, e.g., References 2J and 2K).

If immune suppression is not completely overcome by this treatment, the antigens can be combined in Listeria with a chemotherapeutic agent, such as gemcitabine, that reduces the number of myeloid-derived suppressor cells (MDSCs). MDSCs are the most important contributor to immune suppression in the tumor microenvironment.

Listeria is an exquisitely tumor-specific delivery system for shuttling anticancer agents into tumors and metastases. Its clinical usefulness in delivering therapeutic levels of radioactivity into tumors and metastases has been demonstrated as resulting in a dramatic reduction of advanced pancreatic cancer (See References 3L and 3M). Listeria can be used to deliver childhood vaccine antigens, such as Tetanus Toxoid, as effective recall antigens for pre-existing memory cells (See Reference 3N). Cancer patients, who are more likely to be elderly, are amongst the most vulnerable to SARS-CoV-2, because their immune system is less effective than those of young healthy adults. Therefore, according to the present invention, the SARS-CoV-2 spike protein can be used an antigen in the Listeria-Spike construct. This dual approach fights both cancer and SARS-CoV-2 at the same time (or separately in certain embodiments).

Multiple low doses of Listeria-protein constructs are superior in generating T cell responses to the target antigen, and in reducing metastatic cancer, at old (and young) age (see FIGS. 14A-D). According to the present invention, this is also true for Listeria-Spike. The spike protein is a highly immunogenic protein exhibiting multiple human and mouse B and T cell epitopes. The approach of the present invention invention is effective to fight cancer and viruses, and particularly attractive for elderly cancer patients.

### REFERENCES

The disclosures of the following publications and any other documents or publications cited or otherwise discussed herein, and any file wrappers (e.g., including but not limited to case histories) of any patents or patent applications cited or otherwise discussed herein, and patents or patent applications related thereto and their file wrappers, are hereby incorporated by reference in their entirety into the subject application to more fully describe the art to which the subject invention pertains.

### Reference 1A

The following reference discusses the structure of the spike protein of the SARS-CoV-2 virus: Wrapp D, Wang N, Corbett KS, Goldsmith JA, Hsieh CL, Abiona O, Graham BS, McLellan JS. Cryo-EM structure of the 2019-nCoV spike in the prefusion conformation. Science. 2020 Mar 13; 367(6483):1260-1263. doi: 10.1126/science.abb2507. Epub 2020 Feb 19. PMID: 32075877; PMCID: PMC7164637.

### Reference 1B

The following reference includes the supplementary materials for Reference 1A: Supplementary Materials for Wrapp D, Wang N, Corbett KS, Goldsmith JA, Hsieh CL, Abiona O, Graham BS, McLellan JS. Cryo-EM structure of the 2019-nCoV spike in the prefusion conformation. Science. 2020 Mar 13; 367(6483):1260-1263. doi: 10.1126/science.abb2507. Epub 2020 Feb 19. PMID: 32075877; PMCID: PMC7164637.

### Reference 1C

The following reference includes the sequence of the spike protein of the SARS-CoV-2 virus: Amanat F, Stadlbauer D, Strohmeier S, Nguyen THO, Chromikova V, McMahon M, Jiang K, Arunkumar GA, Jurczyszak D, Polanco J, Bermudez-Gonzalez M, Kleiner G, Aydillo T, Miorin L, Fierer DS, Lugo LA, Kojic EM, Stoever J, Liu STH, Cunningham-Rundles C, Felgner PL, Moran T, García-Sastre A, Caplivski D, Cheng AC, Kedzierska K, Vapalahti O, Hepojoki JM, Simon V, Krammer F. A serological assay to detect SARS-CoV-2 seroconversion in humans. Nat Med. 2020 Jul; 26(7):1033-1036. doi: 10.1038/s41591-020-0913-5. Epub 2020 May 12. PMID: 32398876; PMCID: PMC8183627.

### Reference 1D

The following reference includes the sequence of the spike protein of the SARS-CoV-2 virus: Leibowitz JL, Srinivasa R, Williamson ST, et al. Genetic determinants of mouse hepatitis virus strain 1 pneumovirulence. J Virol. 2010; 84(18):9278-9291. doi:10.1128/JVI.00330-10.

### Reference 1E

The following reference discusses identifying epitopes on the spike protein of the SARS-CoV-2 virus: Zhang BZ, Hu YF, Chen LL, Yau T, Tong YG, Hu JC, Cai JP, Chan KH, Dou Y, Deng J, Wang XL, Hung IF, To KK, Yuen KY, Huang JD. Mining of epitopes on spike protein of SARS-CoV-2 from COVID-19 patients. Cell Res. 2020 Aug; 30(8):702-704. doi: 10.1038/s41422-020-0366-x. Epub 2020 Jul 1. PMID: 32612199; PMCID: PMC7327194.

### Reference 1F

The following reference includes supplementary information for Reference 1F: Supplementary Materials for Zhang BZ, Hu YF, Chen LL, Yau T, Tong YG, Hu JC, Cai JP, Chan KH, Dou Y, Deng J, Wang XL, Hung IF, To KK, Yuen KY, Huang JD. Mining of epitopes on spike protein of SARS-CoV-2 from COVID-19 patients. Cell Res. 2020 Aug; 30(8):702-704. doi: 10.1038/s41422-020-0366-x. Epub 2020 Jul 1. PMID: 32612199; PMCID: PMC7327194.

### Reference 1G

The following reference discusses identifying epitopes on the spike protein of the SARS-CoV-2 virus: Grifoni, Alba et al., A Sequence Homology and Bioinformatic Approach Can Predict Candidate Targets for Immune Responses to SARS-CoV-2, Cell Host & Microbe, Volume 27, Issue 4, 671-680.e2.

### Reference 1H

The following reference discusses identifying epitopes on the spike protein of the SARS-CoV-2 virus: Ethan Fast, Russ B. Altman, Binbin Chen, Potential T-cell and B-cell Epitopes of 2019-nCoV, bioRxiv 2020.02.19.955484; doi: https://doi.org/10.1101/2020.02.19.955484.

### Reference 2A

Achkar JM, Fries BC. 2010. Candida infections of the genitourinary tract. Clinical microbiology reviews 23: 253-73.

### Reference 2B

Eroles P, Sentandreu M, Elorza MV, Sentandreu R. 1997. The highly immunogenic enolase and Hsp70p are adventitious Candida albicans cell wall proteins. Microbiology 143 (Pt 2): 313-20.

### Reference 2C

Lechner MG, Epstein AL. 2011. A new mechanism for blocking myeloid-derived suppressor cells by CpG. Clinical cancer research: an official journal of the American Association for Cancer Research 17: 1645-8.

### Reference 2D

Bracci L, Schiavoni G, Sistigu A, Belardelli F. 2014. Immune-based mechanisms of cytotoxic chemotherapy: implications for the design of novel and rationale-based combined treatments against cancer. Cell Death Differ 21: 15-25.

### Reference 2E

Rice J, Buchan S, Stevenson FK. 2002. Critical components of a DNA fusion vaccine able to induce protective cytotoxic T cells against a single epitope of a tumor antigen. Journal of immunology 169: 3908-13.

### Reference 2F

Reveneau N, Geoffroy MC, Locht C, Chagnaud P, Mercenier A. 2002. Comparison of the immune responses induced by local immunizations with recombinant Lactobacillus plantarum producing tetanus toxin fragment C in different cellular locations. Vaccine 20: 1769-77.

### Reference 2G

Weidinger G, Czub S, Neumeister C, Harriott P, ter Meulen V, Niewiesk S. 2000. Role of CD4(+) and CD8(+) T cells in the prevention of measles virus-induced encephalitis in mice. J Gen Virol 81: 2707-13.

### Reference 2H

Usherwood EJ, Nash AA. 1995. Lymphocyte recognition of picornaviruses. J Gen Virol 76 (Pt 3): 499-508.

### Reference 2I

Chandra D, Jahangir A, Quispe-Tintaya W, Einstein MH, Gravekamp C. 2013. Myeloid-derived suppressor cells have a central role in attenuated Listeria monocytogenes-based immunotherapy against metastatic breast cancer in young and old mice. British Journal of Cancer 108: 2281-2290. Epub 2013 May 2.

### Reference 2J

Quispe-Tintaya W, Chandra D, Jahangir A, Harris M, Casadevall A, Dadachova E, Gravekamp C. 2013. Nontoxic radioactive Listeria(at) is a highly effective therapy against metastatic pancreatic cancer. Proc Natl Acad Sci USA. 21; 110(21):8668-73, Epub 2013 Apr 22.

### Reference 2K

U.S. Patent Application Publication No. 2014/0147379 A1, published May 29, 2014, Dadachova et al., Radiobacteria for Therapy of Cancer.

### Reference 2L

Kim, S.H., Castro, F., Paterson, Y. & Gravekamp, C. 2009. High efficacy of a Listeria-based vaccine against metastatic breast cancer reveals a dual mode of action. Cancer Res 69, 5860-5866.

### Reference 2M

U.S. Patent Application Publication No. 2018/0104320 A1, published April 19, 2018, Gravekamp, Treatment of Cancer Using Recall Antigens Delivered by Attenuated Bacteria.

### Reference 2N

U.S. patent application Ser. No. 15/568,491, filed October 23, 2017, Gravekamp, Treatment of Cancer Using Recall Antigens Delivered by Attenuated Bacteria.

### Reference 3A

Siegel, R.L., Miller, K.D. & Jemal, A. Cancer statistics, 2016. CA Cancer J Clin 66, 7-30, doi:10.3322/caac.21332 (2016).

### Reference 3B

Jemal, A. et al. Global cancer statistics. CA Cancer J Clin 61, 69-90, doi:10.3322/caac.20107 (2011).

### Reference 3C

Sant, M. et al. Survival of women with cancers of breast and genital organs in Europe 1999-2007: Results of the EUROCARE-5 study. Eur J Cancer 51, 2191-2205, doi:10.1016/j.ejca.2015.07.022 (2015).

### Reference 3D

Matulonis, U.A. et al. Ovarian cancer. Nat Rev Dis Primers 2, 16061, doi:10.1038/nrdp.2016.61 (2016).

### Reference 3E

Ford, C.E., Werner, B., Hacker, N.F. & Warton, K. The untapped potential of ascites in ovarian cancer research and treatment. Br J Cancer 123, 9-16, doi:10.1038/s41416-020-0875-x (2020).

### Reference 3F

Pieterse, Z. et al. Ovarian cancer stem cells and their role in drug resistance. Int J Biochem Cell Biol 106, 117-126, doi:10.1016/j.biocel.2018.11.012 (2019).

### Reference 3G

McElhaney, J.E., Meneilly, G.S., Lechelt, K.E. & Bleackley, R.C. Split-virus influenza vaccines: do they provide adequate immunity in the elderly? Journal of gerontology 49, M37-43 (1994).

### Reference 3H

Gravekamp, C. The impact of aging on cancer vaccination. Current opinion in immunology 23, 555-560, doi:10.1016/j.coi.2011.05.003 (2011).

### Reference 3I

Miller, R.A. The aging immune system: primer and prospectus. Science 273, 70-74 (1996).

### Reference 3J

Utsuyama, M. et al. Differential age-change in the numbers of CD4+CD45RA+ and CD4+CD29+ T cell subsets in human peripheral blood. Mechanisms of ageing and development 63, 57-68 (1992).

### Reference 3K

Chandra, D., Jahangir, A., Quispe-Tintaya, W., Einstein, M.H. & Gravekamp, C. Myeloid-derived suppressor cells have a central role in attenuated Listeria monocytogenes-based immunotherapy against metastatic breast cancer in young and old mice. British journal of cancer 108, 2281-2290, doi:10.1038/bjc.2013.206 (2013).

### Reference 3L

Quispe-Tintaya, W. et al. Nontoxic radioactive Listeria(at) is a highly effective therapy against metastatic pancreatic cancer. Proc Natl Acad Sci USA 110, 8668-8673, doi:10.1073/pnas. 1211287110 (2013).

### Reference 3M

Chandra, D. et al. 32-Phosphorus selectively delivered by Listeria to pancreatic cancer demonstrates a strong therapeutic effect. Oncotarget 8, 20729-20740, doi:10.18632/oncotarget.15117 (2017).

### Reference 3N

Selvanesan BC, C.D., Quispe-Tintaya Q, Jahangir, Patel A, Meena K, Alves Da Silva R, Libutti SK, Yuan Z, Beck A, Tesfa L, Koba W, Chuy J, McAuliffe J, Jafari R, Entenberg D, Wang Y, Condeelis J, and Gravekamp C. Tumor-targeted delivery of childhood vaccine recall antigens by attenuated Listeria reduces pancreatic cancer. BioRchiv https://doi.org/10.1101/600106 (2019).

### Reference 3O

Kim, S.H. et al. Mage-b vaccine delivered by recombinant Listeria monocytogenes is highly effective against breast cancer metastases. British journal of cancer 99, 741-749, doi:10.1038/sj.bjc.6604526 (2008).

### Reference 3P

Gunn, G.R. et al. Two Listeria monocytogenes vaccine vectors that express different molecular forms of human papilloma virus-16 (HPV-16) E7 induce qualitatively different T cell immunity that correlates with their ability to induce regression of established tumors immortalized by HPV-16. Journal of immunology 167, 6471-6479 (2001).

### Reference 3Q

Wrapp, D. et al. Cryo-EM structure of the 2019-nCoV spike in the prefusion conformation. Science 367, 1260-1263, doi:10.1126/science.abb2507 (2020).

### Reference 3R

Gravekamp, C. et al. Immunogenicity and protective efficacy of the alpha C protein of group B streptococci are inversely related to the number of repeats. Infect Immun 65, 5216-5221, doi:10.1128/iai.65.12.5216-5221.1997 (1997).

### Reference 3S

Gravekamp, C., Rosner, B. & Madoff, L.C. Deletion of repeats in the alpha C protein enhances the pathogenicity of group B streptococci in immune mice. Infect Immun 66, 4347-4354, doi:10.1128/IAI.66.9.4347-4354.1998 (1998).

### Reference 3T

Walton, J.B. et al. CRISPR/Cas9-derived models of ovarian high grade serous carcinoma targeting Brca1, Pten and Nf1, and correlation with platinum sensitivity. Sci Rep 7, 16827, doi:10.1038/s41598-017-17119-1 (2017).

### Reference 3U

Zhang, S. et al. Genetically Defined, Syngeneic Organoid Platform for Developing Combination Therapies for Ovarian Cancer. Cancer Discov 11, 362-383, doi:10.1158/2159-8290.CD-20-0455 (2021).

### Reference 3V

Paulo Cesar Maciag, Siniša Radulovic, John Rothman, The first clinical use of a live-attenuated Listeria monocytogenes vaccine: A Phase I safety study of Lm-LLO-E7 in patients with advanced carcinoma of the cervix, Vaccine, Volume 27, Issue 30, 2009, Pages 3975-3983, ISSN 0264-410X, https://doi.org/10.1016/j.vaccine.2009.04.041.

### Reference 3W

Tumor-targeted delivery of childhood vaccine recall antigens by attenuated Listeria reduces pancreatic cancer. Benson Chellakkan Selvanesan, Dinesh Chandra, Wilber QuispeTintaya, Arthee Jahangir, Ankur Patel, Kiran Meena, Rodrigo Alberto Alves Da Silva, Madeline Friedman, Steven K Libutti, Ziqiang Yuan, Jenny Li, Sarah Siddiqui, Amanda Beck, Lydia Tesfa, Wade Koba, Jennifer Chuy, John C. McAuliffe, Rojin Jafari, David Entenberg, Yarong Wang, John Condeelis, Vera DesMarais, Vinod Balachandran, Xusheng Zhang, Claudia Gravekamp. bioRxiv 600106; doi: https://doi.org/10.1101/600106

### Reference 3X

Deng, X.; Baker, S.C. (2021). “Coronaviruses: Molecular Biology (Coronaviridae)”. Encyclopedia of Virology: 198-207. doi:10.1016/B978-0-12-814515-9.02550-9. ISBN 9780128145166.

### Reference 3Y

Wang, Yuhang; Grunewald, Matthew; Perlman, Stanley (2020). Coronaviruses: An Updated Overview of Their Replication and Pathogenesis. Coronaviruses. Methods in Molecular Biology. Vol. 2203. pp. 1-29. doi:10.1007/978-1-0716-0900-2_1. ISBN 978-1-0716-0899-9. PMC 7682345. PMID 32833200

### Reference 3Z

Deng, X.; Baker, S.C. (2021). “Coronaviruses: Molecular Biology (Coronaviridae)”. Encyclopedia of Virology: 198-207. doi:10.1016/B978-0-12-814515-9.02550-9. ISBN 9780128145166

### Reference 4A

V'kovski, Philip; Kratzel, Annika; Steiner, Silvio; Stalder, Hanspeter; Thiel, Volker (March 2021). “Coronavirus biology and replication: implications for SARS-CoV-2”. Nature Reviews Microbiology. 19 (3): 155-170. doi:10.1038/s41579-020-00468-6. PMC 7592455. PMID 33116300

### Reference 4B

Flanagan, Katie L.; Best, Emma; Crawford, Nigel W.; Giles, Michelle; Koirala, Archana; Macartney, Kristine; Russell, Fiona; Teh, Benjamin W.; Wen, Sophie CH (2 October 2020). “Progress and Pitfalls in the Quest for Effective SARS-CoV-2 (COVID-19) Vaccines”. Frontiers in Immunology. 11: 579250. doi:10.3389/fimmu.2020.579250. hdl:11343/251733. PMC 7566192. PMID 33123165.

### Reference 4C

Le, Tung Thanh; Cramer, Jakob P.; Chen, Robert; Mayhew, Stephen (October 2020). “Evolution of the COVID-19 vaccine development landscape”. Nature Reviews Drug Discovery. 19 (10): 667-668. doi:10.1038/d41573-020-00151-8. PMID 32887942. S2CID 221503034.

### Reference 4D

Kyriakidis, Nikolaos C.; López-Cortés, Andrés; González, Eduardo Vásconez; Grimaldos, Alejandra Barreto; Prado, Esteban Ortiz (December 2021). “SARS-CoV-2 vaccines strategies: a comprehensive review of phase 3 candidates”. NPJ Vaccines. 6 (1): 28. doi:10.1038/s41541-021-00292-w. PMC 7900244. PMID 33619260.

### Reference 4E (Attached)

NCBI website Accession number OM168995.1.

What is claimed is:

1. A method of treating a subject, comprising administering to the subject an attenuated bacteria that expresses an mRNA-mediated virus vaccination antigen in an amount effective to treat the subject.

2. The method of claim 1, wherein the mRNA-mediated virus vaccination antigen is an epitope of a protein of a virus, the protein being a protein targeted by an mRNA-mediated vaccine against the virus.

3. The method of claim 1, wherein the mRNA-mediated virus vaccination antigen is an epitope of the spike protein of the SARS-CoV-2 virus.

4. The method of claim 1, wherein treating the subject includes treating a tumor in the subject and/or reducing or preventing metastasis of a tumor in the subject, and the amount effective to treat the subject is an amount effective to treat the tumor and/or reduce or prevent metastasis of the tumor.

5. The method of claim 1, wherein treating the subject includes protecting the subject against a virus and the amount effective to treat the subject is an amount effective to prevent infection by the virus and/or eliminate or reduce symptoms of an infection by the virus.

6. The method of claim 5, wherein treating the subject further includes treating a tumor in the subject and/or reducing or preventing metastasis of a tumor in the subject and the amount effective to treat the subject is also an amount effective to treat the tumor and/or reduce or prevent metastasis of the tumor.

7. The method of claim 6, wherein the tumor is a tumor of one or more of the pancreas, ovary, uterus, neck, head, breast, prostate, liver, lung, kidney, neurones, glia, colon, testicle, or bladder.

8. The method of claim 6, wherein the tumor is an inoperable tumor.

9. The method of claim 1, wherein the bacteria is one or more of Listeria monocytogenes, Salmonella thyphimurium, Vibrio cholera, Clostridium, and Bifidobacterium breve.

10. The method of claim 1, wherein prior to administration to the subject, the bacteria are cultured in yeast medium.

11. The method of claim 1, further comprising administering CpG to the subject.

12. The method of claim 1, wherein prior to administration of bacteria to the subject, the subject is screened for their major histocompatibility complex (MHC) 1 haplotype and administered an antigen for which the subject shows a CD8 T cell recall response.

13. The method of claim 1, wherein prior to administration of bacteria to the subject, an epitope of the antigen is administered to the subject to generate memory T cells to the antigen.

14. The method of claim 1, wherein bacteria are administered systemically to the subject.

15. The method of claim 1, wherein bacteria are administered by direct injection to a site in the subject.

16. The method of claim 1, wherein bacteria are administered in myeloid-derived suppressor cells (MDSCs).

17. The method of claim 1, further comprising administering to the subject a chemotherapeutic agent that reduces the number of myeloid-derived suppressor cells (MDSCs).

18. The method of claim 17, wherein the chemotherapeutic agent is gemcitabine.

19. The method of claim 1, wherein the subject is a mammal.

20. The method of claim 1, wherein the subject is a human.
